(19) United States
(12) Patent Application Publication
(10) Pub. No.: US 2019/0112569 A1
(43) Pub. Date: Apr. 18, 2019

(54) In Situ Raman Spectroscopy Systems and Methods for Controlling Process Variables in Cell Cultures

(71) Applicant: Regeneron Pharmaceuticals, Inc., Tarrytown, NY (US)

(72) Inventors: Mark Czeterko, Tarrytown, NY (US); Anthony DeBaise, Tarrytown, NY (US); William Pierce, Tarrytown, NY (US); Matthew Conway, Tarrytown, NY (US)

(21) Appl. No.: 16/160,194

(22) Filed: Oct. 15, 2018

Related U.S. Application Data
(60) Provisional application No. 62/662,322, filed on Apr. 25, 2018.
(60) Provisional application No. 62/572,828, filed on Oct. 16, 2017.

(51) Int. Cl. C12M 1/34; G01N 21/65; C12M 1/36

(52) U.S. Cl. CPC C12M 41/32; C12M 41/48; G01N 21/65

(57) ABSTRACT

The present invention provides in situ Raman spectroscopy methods and systems for monitoring and controlling one or more process variables in a bioreactor cell culture in order to improve product quality and consistency. The methods and systems utilize in situ Raman spectroscopy and chemometric modeling techniques for real-time assessments of cell cultures, combined with signal processing techniques, for precise continuous feedback and model predictive control of cell culture process variables. Through the use of real-time data from Raman spectroscopy, the process variables within the cell culture may be continuously or intermittently monitored and automated feedback controllers maintain the process variables at predetermined set points or maintain a specific feeding protocol that delivers variable amounts of agents to the bioreactor to maximize bioproduct quality.

### CROSS REFERENCE TO RELATED APPLICATIONS

This application claims benefit of and priority to U.S. Provisional Patent Applications 62/572,828 filed on Oct. 16, 2018, and 62/662,322 filed on Apr. 25, 2018, all of which are incorporated by reference in their entireties where permissible.

### FIELD OF THE INVENTION

The invention is generally directed to bioreactor systems and methods including in situ Raman spectroscopy methods and systems for monitoring and controlling one or more process variables in a bioreactor cell culture.

### BACKGROUND OF THE INVENTION

The Process Analytical Technology (PAT) framework of the Food and Drug Administration (FDA) encourages the voluntary development and implementation of innovative solutions for process development, process analysis, and process control to better understand processes and control the quality of products. Process parameters are monitored and controlled during the manufacturing process. For example, the feeding of nutrients to a cell culture in a bioreactor during the manufacturing of bioproducts is an important process parameter. Current bioproduct manufacturing involves a feed strategy of daily bolus feeds. Under current methods, daily bolus feeds increase the nutrient concentration in the cell cultures by at least five times each day. To ensure that the culture is not depleted of nutrients in between feedings, the daily bolus feeds maintain nutrients at high concentration levels. Indeed, each feed is designed to have all of the nutrients that the culture requires to sustain it until the next feed. However, the large amount of nutrients in each daily bolus feed can cause substantial swings in nutrient levels in the bioreactor leading to inconsistencies in the product quality output of the production culture.

In addition, the high concentration of nutrients in each daily bolus feed contributes to an increase in post-translational modifications in the resulting bioproduct. For example, high concentrations of glucose in the cell culture can lead to an increase in glycation in the final bioproduct. Glycation is the nonenzymatic addition of a reducing sugar to an amino acid residue of the protein, typically occurring at the N-terminal amine of proteins and the positively charged amine group. The resulting products of glycation can have yellow or brown optical properties, which can result in colored drug product (Hodge J E (1953) J Agric Food Chem. 1:928-943). Glycation can also result in charge variants within a single production batch of a therapeutic monoclonal antibody (mAb) and result in binding inhibition (Haberger M et al. (2014) MAbs. 6:327-339).

Accordingly, in an effort to further the PAT initiative, there remains a need for a method or system that is able to optimize nutrient concentrations within the cell culture leading to higher quality products.

### SUMMARY OF THE INVENTION

In situ Raman spectroscopy methods and systems for monitoring and controlling one or more process variables in a bioreactor cell culture are disclosed herein.

One embodiment of the present invention includes a method for controlling cell culture medium conditions including quantifying one or more analytes in the cell culture medium using in situ Raman spectroscopy; and adjusting the one or more analyte concentrations in the cell culture medium to match predetermined analyte concentrations that maintain post-translational modifications of proteins in the cell culture medium to 1.0 to 30 percent. In some embodiments, the post-translational modification includes glycation. In other embodiments, proteins in the cell culture include an antibody, antigen-binding fragment thereof, or a fusion protein. In still other embodiments, the cell culture medium includes mammalian cells, for example, Chinese Hamster Ovary cells.

In some embodiments, the analyte is glucose. In this aspect, the predetermined glucose concentration is 0.5 to 8.0 g/L. In another embodiment, the predetermined glucose concentration is 1.0 g/L to 3.0 g/L. In still another embodiment, the glucose concentration is 2.0 g/L or 1.0 g/L. In other embodiments, the predetermined analyte concentrations maintain post-translational modifications of proteins in the cell culture medium to 1.0 to 20 percent or 5.0 to 10 percent. In still other embodiments, the quantifying of analytes is performed continuously, intermittently, or in intervals. For example, the quantifying of analytes is performed in 5 minute intervals, 10 minute intervals, or 15 minute intervals. In yet other embodiments, the quantifying of analytes is performed hourly or at least daily. In some embodiments, the adjusting of analyte concentrations is performed automatically. In still other embodiments, at least two or at least three or at least four different analytes are quantified.

Another embodiment of the present invention includes a method for reducing post-translation modifications of a secreted protein including culturing cells secreting the protein in a cell culture medium including 0.5 to 8.0 g/L glucose; incrementally determining the concentration of glucose in the cell culture medium during culturing of the cells using in situ Raman spectroscopy; and adjusting the glucose concentration to maintain the concentration of glucose to 0.5 to 8.0 g/L by automatically delivering multiple doses of glucose per hour to maintain post-translational modifications of the secreted protein to 1.0 to 30.0 percent. In one embodiment, the concentration of glucose is 1.0 to 3.0 g/L.

Still another embodiment of the present invention includes a system for controlling cell culture medium conditions including one or more processors in communication with a computer readable medium storing software code for execution by the one or more processors in order to cause the system to receive data including a concentration of one or more analytes in the cell culture medium from an in situ Raman spectrometer; and adjust the one or more analyte concentrations in the cell culture medium to match predetermined analyte concentrations that maintain post-translational modifications of proteins in the cell culture medium to 1.0 to 30 percent. In one embodiment, the software code is further configured to cause the system to perform chemometric analysis, for example, Partial Least Squares regression modeling, on the data. In other embodiments, the software code is further configured to cause the system to perform one or more signal processing techniques, for example, a noise reduction technique, on the data.

Another embodiment of the present invention includes a system for reducing post-translation modifications of a secreted protein including one or more processors in communication with a computer readable medium storing software code for execution by the one or more processors in order to cause the system to incrementally receive spectral data including a concentration of glucose in a cell culture medium during culturing of cells secreting the protein from an in situ Raman analyzer; and adjust the glucose concentration to maintain the concentration of glucose to 0.5 to 8.0 g/L, for example, to 1.0 to 3.0 g/L, by automatically delivering multiple doses of glucose per hour to maintain post-translational modifications of the secreted protein to 1.0 to 30.0 percent. In one embodiment, the software code is further configured to cause the system to correlate peaks within the spectral data to glucose concentrations. In another embodiment, the software code is further configured to perform Partial Least Squares regression modeling on the spectral data. In still another embodiment, the software code is further configured to perform a noise reduction technique on the spectral data. In yet other embodiments, the adjustment of the glucose concentration is performed by automated feedback control software.

### BRIEF DESCRIPTION OF THE DRAWINGS

Further features and advantages of the invention can be ascertained from the following detailed description that is provided in connection with the drawings described below:

FIG. 1 is a flow chart of a method for controlling process variables in a cell culture according to one embodiment of the present invention.

FIG. 2 is a schematic diagram of a system for controlling process variables in a cell culture associated with FIG. 1 in accordance with the present invention.

FIG. 3 is a graph showing predicted nutrient process values confirmed by offline nutrient samples.

FIG. 4 is a graph showing filtered final nutrient process values after a signal processing technique according to the present invention.

FIG. 5 is a graph showing the predicted nutrient process values and the filtered final nutrient process values after a shift in the predefined set point of nutrient concentration.

FIG. 6 is a line graph showing the effects of glucose concentration on post-translational modifications for a feedback controlled continuous nutrient feed in accordance with the present invention and for a bolus nutrient feed.

FIG. 7 is a graph showing the in situ Raman predicted glucose concentration values for a feedback controlled continuous nutrient feed in accordance with the present invention and for a bolus nutrient feed.

FIG. 8 is a line graph showing the antibody titer for a feedback controlled continuous nutrient feed in accordance with the present invention and for a bolus nutrient feed.

FIG. 9 is a bar graph showing shows the normalized percentage of post-translational modifications as a result of glucose concentration.

FIG. 10 is a graph showing the glucose concentrations for a feedback controlled continuous nutrient feed in accordance with the present invention and for a bolus nutrient feed.

FIG. 11 is a graph showing that feedback control cell culture can reduce the PTMs by as much as 50% compared to bolus fed strategy cell culture.

### DETAILED DESCRIPTION

### I. Definitions

As used herein, the singular forms “a,” “an,” and “the” include plural referents unless the context clearly dictates otherwise.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments, the values may range in value either above or below the stated value in a range of approx. +/−5%; in other embodiments, the values may range in value either above or below the stated value in a range of approx. +/−2%; in other embodiments, the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The term “bioproduct” refers to any antibody, antibody fragment, modified antibody, protein, glycoprotein, or fusion protein as well as final drug substances manufactured in a bioreactor process.

The terms “control” and “controlling” refer to adjusting an amount or concentration level of a process variable in a cell culture to a predefined set point.

The terms “monitor” and “monitoring” refer to regularly checking an amount or concentration level of a process variable in a cell culture or a process condition in the cell culture.

The term “steady state” refers to maintaining the concentration of nutrients, process parameters, or the quality attributes in the cell culture at an unchanging, constant, or stable level.

It is understood that an unchanging, constant, or stable level refers to a level within predetermined set points. Set points, and therefore steady state levels, may be shifted during the time period of a production cell culture by the operator.

### II. Methods for Producing Bioproducts

One embodiment provides methods for monitoring and controlling one or more process variables in a bioreactor cell culture in order to improve product quality and consistency. Process variables include but are not limited to concentrations of glucose, amino acids, vitamins, growth factors, proteins, viable cell count, oxygen, nitrogen, pH, dead cell count, cytokines, lactate, glutamine, other sugars such as fructose and galactose, ammonium, osmolality, and combinations thereof. The disclosed methods and systems utilize in situ Raman spectroscopy and chemometric modeling techniques for real-time assessments of cell cultures, combined with signal processing techniques, for precise continuous feedback and model predictive control of cell culture process variables. In situ Raman spectroscopy of the bioreactor contents allows the analysis of one or more process variables in the bioreactor without having to physically remove a sample of the bioreactor contents for testing. Through the use of real-time data from Raman spectroscopy, the process variables within the cell culture may be continuously or intermittently monitored and automated feedback controllers maintain the process variables at predetermined set points or maintain a specific feeding protocol that delivers variable amounts of agents to the bioreactor to maximize bioproduct quality.

The disclosed methods and systems control one or more process variables in a cell culture process. The terms, “cell culture” and “cell culture media,” may be used interchangeably and include any solid, liquid or semi-solid designed to support the growth and maintenance of microorganisms, cells, or cell lines. Components such as polypeptides, sugars, salts, nucleic acids, cellular debris, acids, bases, pH buffers, oxygen, nitrogen, agents for modulating viscosity, amino acids, growth factors, cytokines, vitamins, cofactors, and nutrients may be present within the cell culture medium. One embodiment provides a mammalian cell culture process and include mammalian cells or cell lines. For example, a mammalian cell culture process may utilize a Chinese Hamster Ovary (CHO) cell line grown in a chemically defined basal medium.

The cell culture process may be performed in a bioreactor. The bioreactors include seed train, fed-batch, and continuous bioreactors. The bioreactors may range in volume from about 2 L to about 10,000 L. In one embodiment, the bioreactor may be a 60 L stainless steel bioreactor. In another embodiment, the bioreactor may be a 250 L bioreactor. Each bioreactor should also maintain a cell count in the range of about 5×10⁶ cells/mL to about 100×10⁶ cells/mL. For example, the bioreactor should maintain a cell count of about 20×10⁶ cells/mL to about 80 cells/mL.

The disclosed methods and system can monitor and control any analyte that is present in the cell culture and has a detectable Raman spectrum. For example, the methods of the present invention may be used to monitor and control any component of the cell culture media including components added to the cell culture, substances secreted from the cell, and cellular components present upon cell death. Components of the cell culture media that may be monitored and/or controlled by the disclosed systems and methods include, but are not limited to, nutrients, such as amino acids and vitamins, lactate, co-factors, growth factors, cell growth rate, pH, oxygen, nitrogen, viable cell count, acids, bases, cytokines, antibodies, and metabolites.

One embodiment provides the methods for monitoring and controlling nutrient concentrations in a cell culture. As used herein, the term “nutrient” may refer to any compound or substance that provides nourishment essential for growth and survival. Examples of nutrients include, but are not limited to, simple sugars such as glucose, galactose, lactose, fructose, or maltose; amino acids; and vitamins, such as vitamin A, B vitamins, and vitamin E. In another embodiment, the methods of the present invention may include monitoring and controlling glucose concentrations in a cell culture. By controlling the nutrient concentrations, for example, glucose concentrations, in a cell culture, it has been discovered that bioproducts, such as proteins, can be produced in a lower concentration range than was previously possible using a daily bolus nutrient feeding strategy.

Moreover, by controlling nutrient concentrations and other process variables in the cell culture, the methods of the present invention further provide for modulating one or more post-translational modifications of a protein. Without being bound by any particular theory, it is believed that, by providing lower nutrient concentrations within the cell culture, post-transitional modifications in proteins and antibodies may be decreased. Examples of post-translational modifications that may be modulated by the present invention include, but are not limited to, glycation, glycosylation, acetylation, phosphorylation, amidation, derivatization by known protecting/blocking groups, proteolytic cleavage, and modification by non-naturally occurring amino acids. Another embodiment provides methods and systems for modulating the glycation of a protein. For instance, by providing lower concentration ranges of glucose in cell culture media, levels of glycation in secreted protein or antibody can be decreased in the final bioproduct.

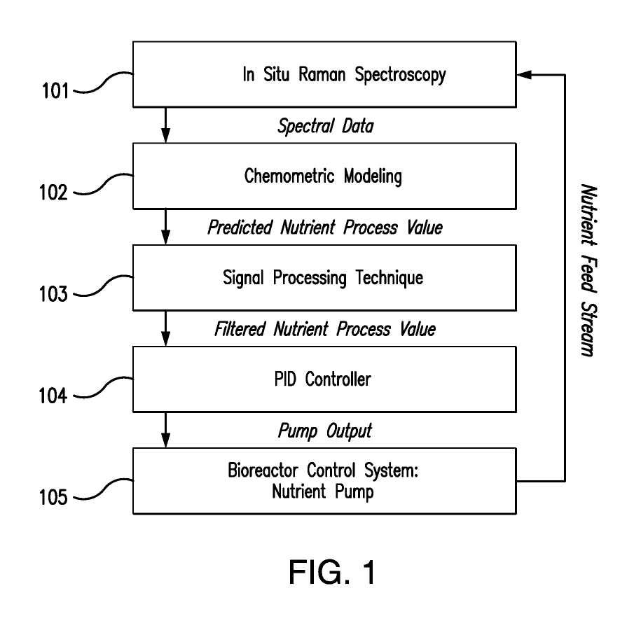

FIG. 1 is a flow chart of an exemplary method for controlling one or more process variables, for example, nutrient concentration, in a bioreactor cell culture. Predetermined set points for each of the process variables to be monitored and controlled can be programmed into the system. The predefined set points represent the amount of process variable in the cell culture that is to be maintained or adjusted throughout the process. Glucose concentration is one example of a nutrient that can be monitored and modulated. As briefly discussed above, it has been discovered that bioproducts (for example, proteins, antibodies, fusion proteins, and drug substances) can be produced by cells in a culture medium that contains low levels of glucose compared to glucose concentrations in media using a daily bolus nutrient feeding strategy. In one embodiment, the predefined set point for nutrient concentration is the lowest concentration of a nutrient necessary to grow and propagate a cell line. The disclosed methods and systems can deliver multiple small doses of nutrients to the culture medium over a period of time or can provide a steady stream of nutrient to the culture medium. In some embodiments, the predefined set point may be increased or decreased during the process depending on the conditions within the cell culture media. For example, if the predefined amount of nutrient concentration results in cell death or sub-optimal growth conditions within the cell culture media, the predefined set point may be increased. However, the nutrient concentration should be maintained at a predefined set point of about 0.5 g/L to about 10 g/L. In another embodiment, the nutrient concentration should be maintained at a predefined set point of about 0.5 g/L to about 8 g/L. In still another embodiment, the nutrient concentration should be maintained at a predefined set point of about 1 g/L to about 3 g/L. In yet another embodiment, the nutrient concentration should be maintained at a predefined set point of about 2 g/L. These predefined set points essentially provide a baseline level at which the nutrient concentration should be maintained throughout the process.

In one embodiment, the monitoring of the one or more process variables, for example, the nutrient concentration, in a cell culture is performed by Raman spectroscopy (step 101). Raman spectroscopy is a form of vibrational spectroscopy that provides information about molecular vibrations that can be used for sample identification and quantitation. In some embodiments, the monitoring of the process variables is performed using in situ Raman spectroscopy. In situ Raman analysis is a method of analyzing a sample in its original location without having to extract a portion of the sample for analysis in a Raman spectrometer. In situ Raman analysis is advantageous in that the Raman spectroscopy analyzers are noninvasive, which reduces the risk of contamination, and nondestructive with no impact to cell culture viability or protein quality.

The in situ Raman analysis can provide real-time assessments of one or more process variables in cell cultures. For example, the raw spectral data provided by in situ Raman spectroscopy can be used to obtain and monitor the current amount of nutrient concentration in a cell culture. In this aspect, to ensure that the raw spectral data is continuously up to date, the spectral data from the Raman spectroscopy should be acquired about every 10 minutes to 2 hours. In another embodiment, the spectral data should be acquired about every 15 minutes to 1 hour. In still another embodiment, the spectral data should be acquired about every 20 minutes to 30 minutes.

In this aspect, the monitoring of the one or more process variables in the cell culture can be analyzed by any commercially available Raman spectroscopy analyzer that allows for in situ Raman analysis. The in situ Raman analyzer should be capable of obtaining raw spectral data within the cell culture (for example, the Raman analyzer should be equipped with a probe that may be inserted into the bioreactor). Suitable Raman analyzers include, but are not limited to, RamanRXN2 and RamanRXN4 analyzers (Kaiser Optical Systems, Inc. Ann Arbor, Mich.).

In step 102, the raw spectral data obtained by in situ Raman spectroscopy may be compared to offline measurements of the particular process variable to be monitored or controlled (for example, offline nutrient concentration measurements) in order to correlate the peaks within the spectral data to the process variable. For instance, if the process variable to be monitored or controlled is glucose concentration, offline glucose concentration measurements may be used to determine which spectral regions exhibit the glucose signal. The offline measurement data may be collected through any appropriate analytical method. Additionally, any type of multivariate software package, for example, SIMCA 13 (MKS Data Analytic Solutions, Umea, Sweden), may be used to correlate the peaks within the raw spectral data to offline measurements of the particular process variable to be monitored or controlled. However, in some embodiments, it may be necessary to pretreat the raw spectral data with spectral filters to remove any varying baselines. For example, the raw spectral data may be pretreated with any type of point smoothing technique or normalization technique. Normalization may be needed to correct for any laser power variation and exposure time by the Raman analyzer. In one embodiment, the raw spectral data may be treated with point smoothing, such as 1^{st} derivative with 21 cm⁻¹ point smoothing, and normalization, such as Standard Normal Variate (SNV) normalization.

Chemometric modeling may also be performed on the obtained spectral data. In this aspect, one or more multivariate methods including, but not limited to, Partial Least Squares (PLS), Principal Component Analysis (PCA), Orthogonal Partial least squares (OPLS), Multivariate Regression, Canonical Correlation, Factor Analysis, Cluster Analysis, Graphical Procedures, and the like, can be used on the spectral data. In one embodiment, the obtained spectral data is used to create a PLS regression model. A PLS regression model may be created by projecting predicted variables and observed variables to a new space. In this aspect, a PLS regression model may be created using the measurement values obtained from the Raman analysis and the offline measurement values. The PLS regression model provides predicted process values, for example, predicted nutrient concentration values.

After chemometric modeling, a signal processing technique may be applied to the predicted process values (for example, the predicted nutrient concentration values) (step 103). In one embodiment, the signal processing technique includes a noise reduction technique. In this aspect, one or more noise reduction techniques may be applied to the predicted process values. Any noise reduction technique known to those skilled in the art may be utilized. For example, the noise reduction technique may include data smoothing and/or signal rejection. Smoothing is achieved through a series of smoothing algorithms and filters while signal rejection uses signal characteristics to identify data that should not be included in the analyzed spectral data. In one embodiment, the predicted process values are noise mitigated by a noise reduction filter. The noise reduction filter provides final filtered process values (for example, final filtered nutrient concentration values). In this aspect, the noise reduction technique combines raw measurements with a model-based estimate for what the measurement should yield according to the model. In one embodiment, the noise reduction technique combines a current predicted process value with its uncertainties. Uncertainties can be determined by the repeatability of the predicted process values and the current process conditions. Once the next predicted process value is observed, the estimate of the predicted process value (for example, predicted nutrient concentration value) is updated using a weighted average where more weight is given to the estimates with higher certainty. Using an iterative approach, the final process values may be updated based on the previous measurement and the current process conditions. In this aspect, the algorithm should be recursive and able to run in real time so as to utilize the current predicted process value, the previous value, and experimentally determined constants. The noise reduction technique improves the robustness of the measurements received from the Raman analysis and the PLS predictions by reducing noise upon which the automated feedback controller will act.

Upon obtaining the final filtered process values (for example, the final filtered nutrient concentration values), the final values may be sent to an automated feedback controller (step 104). The automated feedback controller may be used to control and maintain the process variable (for example, the nutrient concentration) at the predefined set point. The automated feedback controller may include any type of controller that is able to calculate an error value as the difference between a desired set point (e.g., the predefined set point) and a measured process variable and automatically apply an accurate and responsive correction. The automated feedback controller should also have controls that are capable of being changed in real time from a platform interface. For instance, the automated feedback controller should have a user interface that allows for the adjustment of a predefined set point. The automated feedback controller should be capable of responding to a change in the predefined set point.

In one embodiment, the automated feedback controller may be a proportional-integral-derivative (PID) controller. In this aspect, the PID controller is operable to calculate the difference between the predefined set point and the measured process variable (for example, the measured nutrient concentration) and automatically apply an accurate correction. For example, when a nutrient concentration of a cell culture is to be controlled, the PID controller may be operable to calculate a difference between a filtered nutrient value and a predefined set point and provide a correction in nutrient amount. In this aspect, the PID controller may be operatively connected to a nutrient pump on the bioreactor so that the corrective nutrient amount may be pumped into the bioreactor (step 105).

Through the use of Raman real time analysis and feedback control, the methods of the present invention are able to provide continuous and reduced concentrations of nutrients to the cell culture. That is, the method of the present invention is able to provide steady-state nutrient addition to the cell culture. In one embodiment, in order to maintain the predefined nutrient concentration, the nutrients may be pumped to the cell culture, via the nutrient pump, continuously over a period of time. In another embodiment, the nutrients may be added to the cell culture, via the nutrient pump, in a duty cycle. For instance, in this aspect, the addition of the nutrients may be staggered or occur intermittently over a period of time.

The disclosed methods and systems also allow for the production of bioproducts in culture media that contains lower nutrient concentration range, for example, glucose concentration range, than nutrient concentrations in culture media using a daily bolus nutrient feeding strategy. In one embodiment, the nutrient concentrations, for example, glucose concentrations, are at least 3 g/L lower than bolus nutrient feedings. In another embodiment, the nutrient concentrations, for example, glucose concentrations, are at least 5 g/L lower than nutrient concentrations in culture media obtained using bolus nutrient feedings. In still another embodiment, the nutrient concentrations, for example, glucose concentrations, are at least 6 g/L lower than nutrient concentrations obtained using bolus nutrient feedings.

Moreover, the lower nutrient concentrations in culture media and steady-state addition achieved by the disclosed systems and methods allow for a decrease in post-translational modification in proteins and monoclonal antibodies. In one embodiment, the disclosed methods and systems deliver nutrients near or at the rate the nutrients are taken up or consumed by cells in the culture. The steady-state addition of small doses of nutrients over time allows for the production of bioproducts having lower levels of post-translational modifications, for example, lower levels of glycation, in comparison to standard bolus feed addition. Importantly, the steady-state addition of the reduced concentrations of nutrients does not affect antibody production. In one embodiment, the reduced nutrient concentrations provide for a decrease in post-translation modification by as much as 30% when compared to the post-translation modifications observed in standard bolus feed addition. In another embodiment, the reduced nutrient concentrations provide for a decrease in post-translation modification by as much as 40% when compared to the post-translation modifications observed in standard bolus feed addition. In still another embodiment, the reduced nutrient concentrations provide for a decrease in post-translation modification by as much as 50% when compared to the post-translation modifications observed in standard bolus feed addition.

### III. Bioreactor Systems

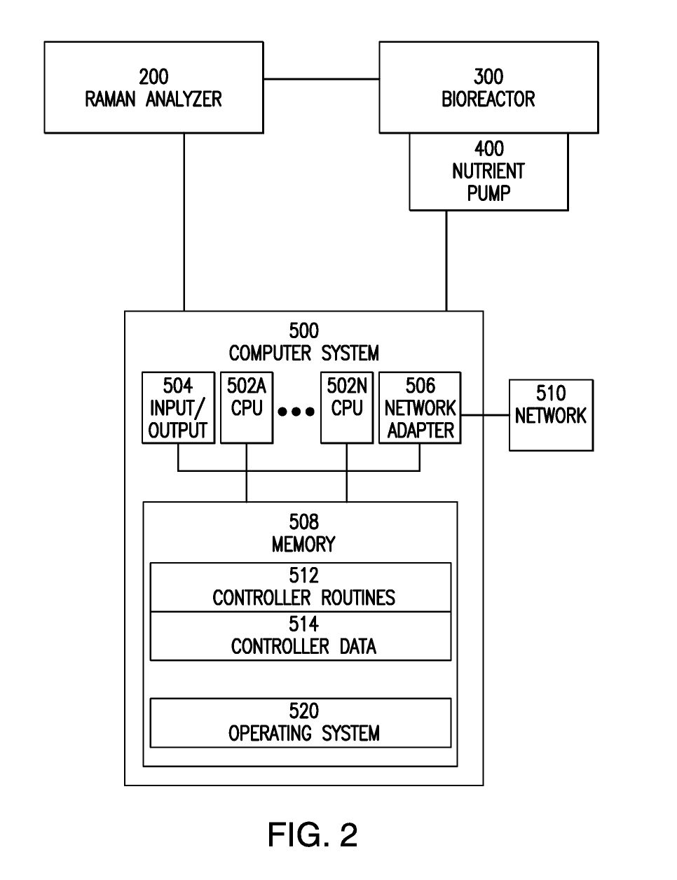

Another embodiment provides systems for monitoring and controlling one or more process variables in a bioreactor cell culture. Multiple components are integrated into a single system with a single user interface. Referring to FIG. 2, Raman analyzer 200 may be operatively connected to bioreactor 300. In this aspect, a Raman probe may be inserted into the bioreactor 300 to obtain raw spectral data of one or more process variables, for example, nutrient concentration, within the cell culture. The Raman analyzer 200 may also be operatively connected to computer system 500 so that the obtained raw spectral data may be received and processed.

Computer system 500 may typically be implemented using one or more programmed general-purpose computer systems, such as embedded processors, systems on a chip, personal computers, workstations, server systems, and minicomputers or mainframe computers, or in distributed, networked computing environments. Computer system 500 may include one or more processors (CPUs) 502A-502N, input/output circuitry 504, network adapter 506, and memory 508. CPUs 502A-502N execute program instructions in order to carry out the functions of the present systems and methods. Typically, CPUs 502A-502N are one or more microprocessors, such as an INTEL CORE® processor.

Input/output circuitry 504 provides the capability to input data to, or output data from, computer system 500. For example, input/output circuitry may include input devices, such as keyboards, mice, touchpads, trackballs, scanners, analog to digital converters, etc., output devices, such as video adapters, monitors, printers, etc., and input/output devices, such as, modems, etc. Network adapter 506 interfaces device 500 with a network 510. Network 510 may be any public or proprietary LAN or WAN, including, but not limited to the Internet.

Memory 508 stores program instructions that are executed by, and data that are used and processed by, CPU 502 to perform the functions of computer system 500. Memory 508 may include, for example, electronic memory devices, such as random-access memory (RAM), read-only memory (ROM), programmable read-only memory (PROM), electrically erasable programmable read-only memory (EEPROM), flash memory, etc., and electro-mechanical memory, such as magnetic disk drives, tape drives, optical disk drives, etc., which may use an integrated drive electronics (IDE) interface, or a variation or enhancement thereof, such as enhanced IDE (EIDE) or ultra-direct memory access (UDMA), or a small computer system interface (SCSI) based interface, or a variation or enhancement thereof, such as fast-SCSI, wide-SCSI, fast and wide-SCSI, etc., or Serial Advanced Technology Attachment (SATA), or a variation or enhancement thereof, or a fiber channel-arbitrated loop (FC-AL) interface.

Memory 508 may include controller routines 512, controller data 514, and operating system 520. Controller routines 512 may include software routines to perform processing to implement one or more controllers. Controller data 514 may include data needed by controller routines 512 to perform processing. In one embodiment, controller routines 512 may include multivariate software for performing multivariate analysis, such as PLS regression modeling. In this aspect, controller routines 512 may include SIMCA-QPp (MKS Data Analytic Solutions, Umea, Sweden) for performing chemometric PLS modeling. In another embodiment, controller routines 512 may also include software for performing noise reduction on a data set. In this aspect, the controller routines 512 may include MATLAB Runtime (The Mathworks Inc., Natick, Mass.) for performing noise reduction filter models. Moreover, controller routines 512 may include software, such as MATLAB Runtime, for operating the automated feedback controller, for example, the PID controller. The software for operating the automated feedback controller should be able to calculate the difference between the predefined set point and the measured process variable (for example, the measured nutrient concentration) and automatically apply an accurate correction. Accordingly, the computer system 500 may also be operatively connected to nutrient pump 400 so that the corrective nutrient amount may be pumped into the bioreactor 300.

The disclosed systems may control and monitor process variables in a single bioreactor or a plurality of bioreactors. In one embodiment, the system may control and monitor process variables in at least two bioreactors. In another embodiment, the system may control and monitor process variables in at least three bioreactors or at least four bioreactors. For example, the system can monitor up to four bioreactors in an hour.

### EXAMPLES

The following non-limiting examples demonstrate methods for controlling one or more process variables in a bioreactor cell culture in accordance with the present invention. The examples are merely illustrative of the preferred embodiments of the present invention, and are not to be construed as limiting the invention, the scope of which is defined by the appended claims.

### Example 1

Materials and Methods

The mammalian cell culture process utilized a Chinese Hamster Ovary (CHO) cell line grown in a chemically defined basal medium. The production was performed in a 60 L pilot scale stainless steel bioreactor controlled by RSLogix 5000 software (Rockwell Automation, Inc. Milwaukee, Wis.).

The data collection for the model included spectral data from both Kaiser RamanRXN2 and RamanRXN4 analyzers (Kaiser Optical Systems, Inc. Ann Arbor, Mich.) utilizing BIO-PRO optic (Kaiser Optical Systems, Inc. Ann Arbor, Mich.). The RamanRXN2 and RamanRXN4 analyzers operating parameters were set to a 10 second scan time for 75 accumulations. An OPC Reader/Writer to RSLinx OPC Server was used for data flow.

SIMCA 13 (MKS Data Analytic Solutions, Umea, Sweden) was used to correlate peaks within the spectral data to offline glucose measurements. The following spectral filtering was performed on the raw spectral data: 1st derivative with 21^{cm-1} point smoothing to remove varying baselines and Standard Normal Variate (SNV) normalization to correct for laser power variation and exposure time.

A Partial Least Squares regression model was created with corresponding offline measurements taken on the Nova Bioprofile Flex (Nova Biomedical, Waltham, Mass.). Table 1A below shows the details of the nutrient chemometric Partial Least Squares regression model.

TABLE 1A
NUTRIENT CHEMOMETRIC PARTIAL LEAST
SQUARES REGRESSION MODEL DETAILS

| Nutrient PLS Model Variable | Value |
|---|---|
| Observations | 223 |
| Wavelength Range (cm⁻¹) | 350-3100 |
| Nutrient Concentration Range (g/L) | 0.65-8.63 |
| RMSEE | 0.430 |
| RSMECV | 0.662 |
| R²X | 0.982 |
| Q² | 0.869 |

Signal processing techniques, specifically, noise reduction filtering, were also performed. The noise reduction technique combined the raw measurement with a model-based estimate for what the measurement should yield according to the model. Using an iterative approach, it allows for the filtered measurement to be updated based on the previous measurement and the current process conditions.

A reverse-acting proportional-integral-derivative (PID) Control having an algorithm programmed separately in MATLAB Runtime (The Mathworks Inc., Natick, Mass.) was utilized. All variables of the PID controller, such as tuning constants, have the ability to be changed in real time from the platform interface.

Results

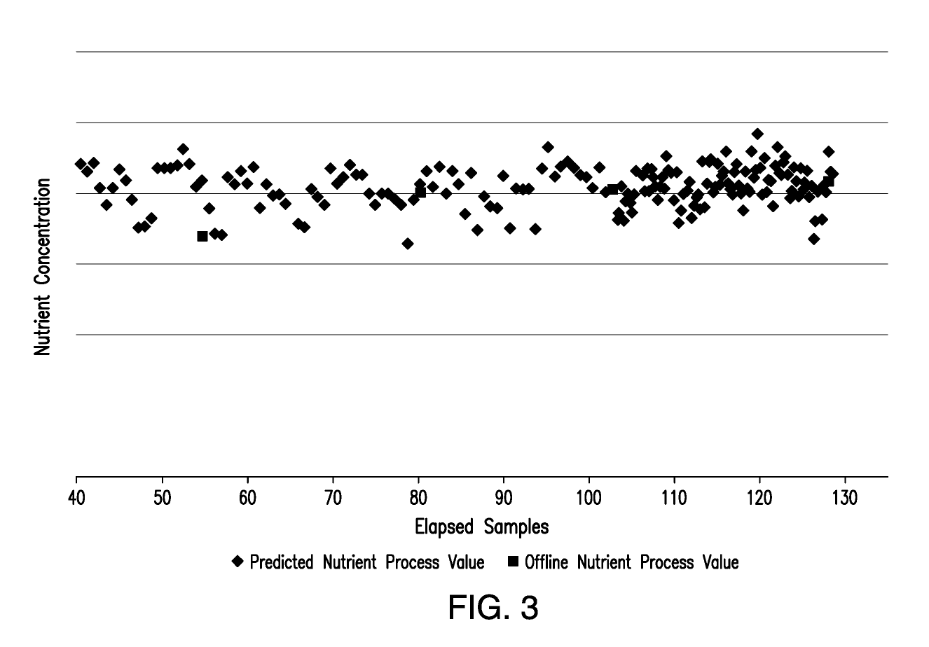

FIG. 3 shows the predicted nutrient process values confirmed by offline nutrient samples. As can be seen from FIG. 3, the Raman analyzer and the chemometric model predicted nutrient concentration values within the offline analytical method's variability. This demonstrates that in situ Raman spectroscopy and chemometric modeling according to the methods of the present invention provide accurate measurements of nutrient concentration values.

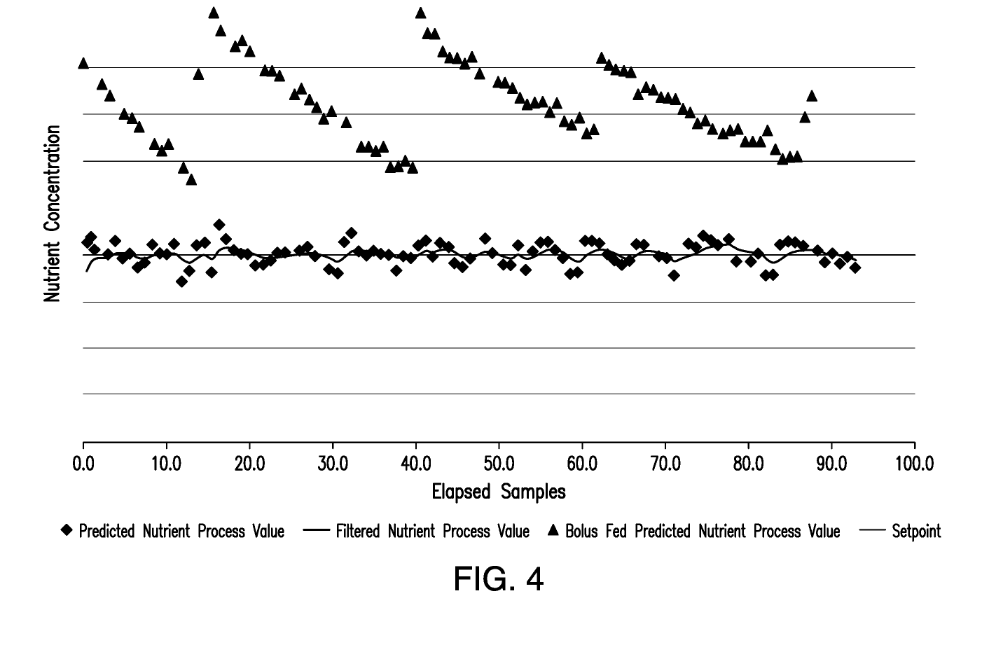

FIG. 4 shows the filtered final nutrient process values after the signal processing technique. As can be seen from FIG. 4, the signal processing technique reduces noise of raw predicted nutrient process values. The noise reduction filtering of the predicted nutrient values increases the robustness of the overall feedback control system.

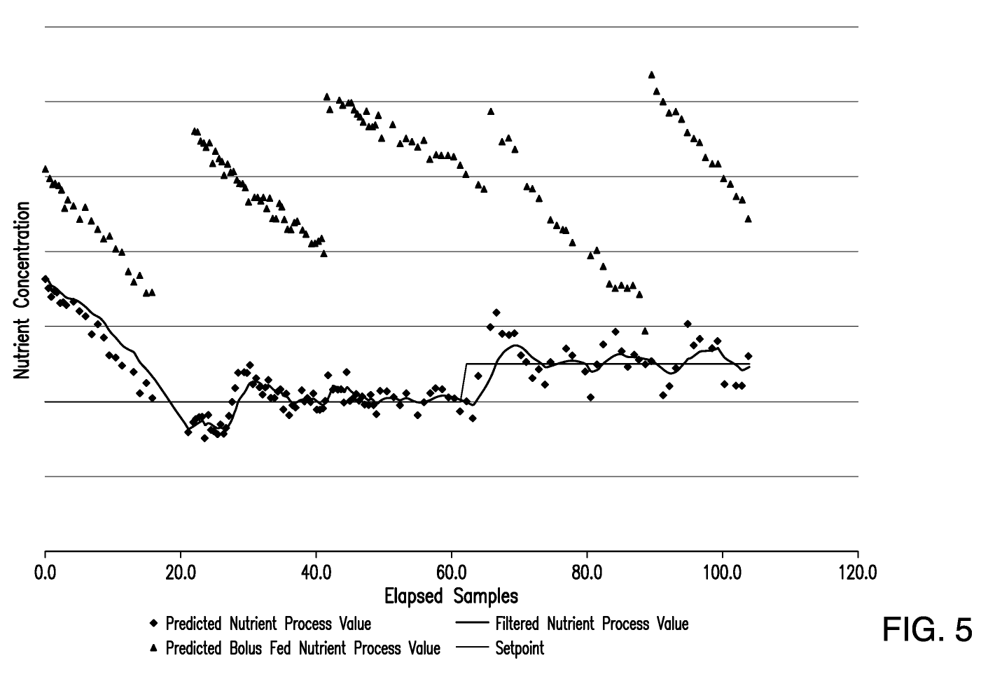

FIG. 5 shows the predicted nutrient process values and the filtered final nutrient process values after a shift in the predefined set point of nutrient concentration in a feedback controlled continuous nutrient feed batch. As can be seen by the adjustment in filtered nutrient process values, a successful response from the feedback controller is observed when a shift in nutrient concentration set point occurs. Indeed, the PID controller was able to quickly respond to a set point change operating off the noise filtered nutrient process value.

Based on the results shown in FIGS. 3-5, the methods of the present invention provide real time data that enables automated feedback control for continuous and steady nutrient addition.

### Example 2

Materials and Methods

The production was performed in 250 L single use bioreactors. A Partial Least Squares regression model was created. Table 1B below shows the details of the nutrient chemometric Partial Least Squares regression model.

TABLE 1B
NUTRIENT CHEMOMETRIC PARTIAL LEAST
SQUARES REGRESSION MODEL DETAILS

| Nutrient PLS Model Variable | Value |
|---|---|
| Observations | 147 |
| Wavelength Range (cm⁻¹) | 350-3100 |
| Nutrient Concentration Range (g/L) | 0.6-3.61 |
| RMSEE | 0.352 |
| RSMECV | 0.520 |
| R²X | 0.769 |
| Q² | 0.617 |

Noise filtering techniques were not used in this example.

Results

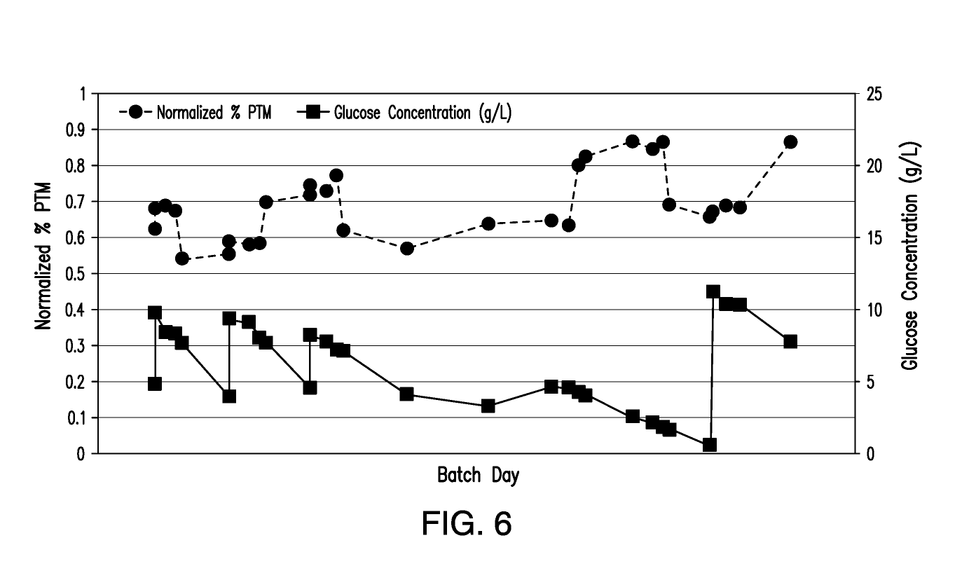

FIG. 6 shows the effects of glucose concentration on post-translational modifications. As can be seen from FIG. 6, the greater the glucose concentration, the higher the percentage of PTM. The data points in FIG. 6 for normalized % of post-translational modification (PTM) and glucose concentration over the hatch day are shown in Table 2 below

TABLE 2
NORMALIZED % PTM AND GLUCOSE CONCENTRATION
DATA POINTS FOR FIG. 6

| | | | | Glucose |
|---|---|---|---|---|
| Time | % | Glucose | Normalized | Concentration |
| (hours) | PTM | Concentration | % PTM | (g/L) |
| 192 | 18.7 | 4.83 | 0.623333333 | 4.83 |
| 192 | 20.4 | 9.75 | 0.68 | 9.75 |
| 195 | 20.6 | 8.4 | 0.686666667 | 8.4 |
| 198 | 20.2 | 8.3 | 0.673333333 | 8.3 |
| 200 | 16.2 | 7.68 | 0.54 | 7.68 |
| 214 | 16.6 | 3.96 | 0.553333333 | 3.96 |
| 214 | 17.7 | 9.34 | 0.59 | 9.34 |
| 220 | 17.4 | 9.09 | 0.58 | 9.09 |
| 223 | 17.5 | 8.03 | 0.583333333 | 8.03 |
| 225 | 20.9 | 7.68 | 0.696666667 | 7.68 |
| 238 | 21.5 | 4.56 | 0.716666667 | 4.56 |
| 238 | 22.3 | 8.22 | 0.743333333 | 8.22 |
| 243 | 21.8 | 7.78 | 0.726666667 | 7.78 |
| 246 | 23.1 | 7.19 | 0.77 | 7.19 |
| 248 | 18.6 | 7.08 | 0.62 | 7.08 |
| 267 | 17 | 4.11 | 0.566666667 | 4.11 |
| 291 | 19.1 | 3.3 | 0.636666667 | 3.3 |
| 310 | 19.4 | 4.62 | 0.646666667 | 4.62 |
| 315 | 19 | 4.55 | 0.633333333 | 4.55 |
| 318 | 24 | 4.23 | 0.8 | 4.23 |
| 320 | 24.7 | 4 | 0.823333333 | 4 |
| 334 | 26 | 2.53 | 0.866666667 | 2.53 |
| 340 | 25.3 | 2.15 | 0.843333333 | 2.15 |
| 343 | 25.9 | 1.86 | 0.863333333 | 1.86 |
| 345 | 20.7 | 1.67 | 0.69 | 1.67 |
| 357 | 19.7 | 0.59 | 0.656666667 | 0.59 |
| 358 | 20.2 | 11.18 | 0.673333333 | 11.18 |
| 362 | 20.6 | 10.34 | 0.686666667 | 10.34 |
| 366 | 20.5 | 10.31 | 0.683333333 | 10.31 |
| 381 | 25.9 | 7.74 | 0.863333333 | 7.74 |

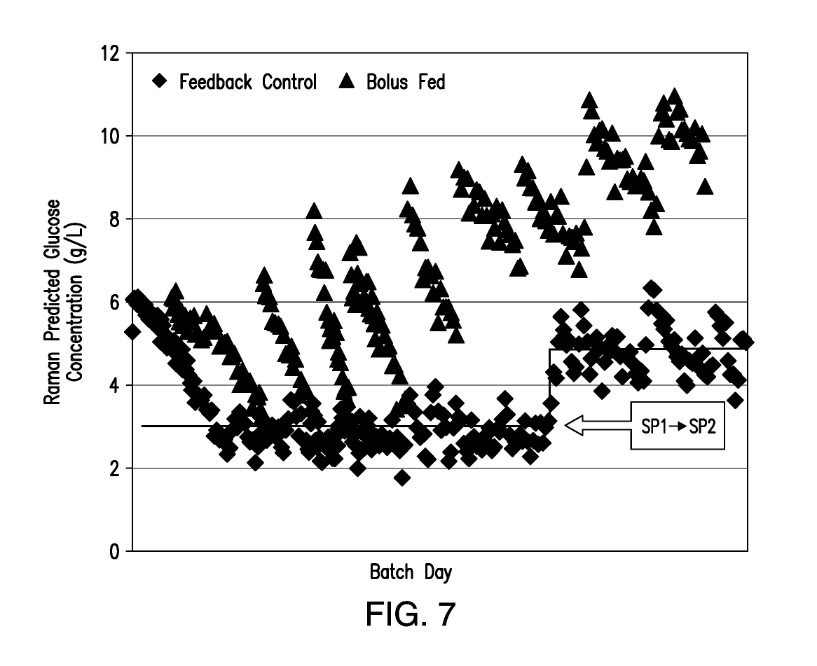

FIG. 7 shows the in situ Raman predicted glucose concentration values for a feedback controlled continuous nutrient feed in accordance with the present invention and for a bolus nutrient feed. The bolded black line in FIG. 7 represents the pre-defined set point. The pre-defined set point (SP1) was initially set at 3 g/L (SP1) and was increased to 5 g/L (SP2). As can be seen from FIG. 7, the Raman predicted glucose concentrations accurately adjusted during a shift in pre-defined set points. The data points in FIG. 7 for the Raman predicted glucose concentration values over the batch day are shown in Table 3 below.

TABLE 3
RAMAN PREDICTED GLUCOSE CONCENTRATION
DATA POINTS FOR FIG. 7

| | Raman Glucose | | Raman |
|---|---|---|---|
| Time (Glucose | Feedback | Time (Glucose | Bolus Feed |
| Feedback Control) | Concentration | Bolus Feed) | Concentration |
| (Elapsed Days) | (g/L) | (Elapsed Days) | (g/L) |
| 2 | 5.27449 | 2 | #N/A |
| 2.023263889 | 6.057528 | 2.023263889 | #N/A |
| 2.044097222 | 6.093102 | 2.044097222 | #N/A |
| 2.064930556 | 6.030814 | 2.064930556 | #N/A |
| 2.085763889 | 5.928053 | 2.085763889 | #N/A |
| 2.106597222 | 6.112341 | 2.106597222 | #N/A |
| 2.127430556 | 5.877689 | 2.127430556 | #N/A |
| 2.148263889 | 5.881066 | 2.148263889 | #N/A |
| 2.169097222 | 5.929256 | 2.169097222 | #N/A |
| 2.189930556 | 5.928593 | 2.189930556 | #N/A |
| 2.210763889 | 5.929407 | 2.210763889 | #N/A |
| 2.231597222 | 5.672209 | 2.231597222 | #N/A |
| 2.252430556 | 5.796999 | 2.252430556 | #N/A |
| 2.273263889 | 5.572541 | 2.273263889 | #N/A |
| 2.294097222 | 5.771776 | 2.294097222 | #N/A |
| 2.31494213 | 5.521614 | 2.31494213 | #N/A |
| 2.335775463 | 5.630873 | 2.335775463 | #N/A |
| 2.356608796 | 5.53435 | 2.356608796 | #N/A |
| 2.37744213 | 5.628556 | 2.37744213 | #N/A |
| 2.398275463 | 5.575116 | 2.398275463 | #N/A |
| 2.419108796 | 5.675688 | 2.419108796 | #N/A |
| 2.43994213 | 5.356216 | 2.43994213 | #N/A |
| 2.460775463 | 5.019809 | 2.460775463 | #N/A |
| 2.481608796 | 5.571718 | 2.481608796 | #N/A |
| 2.50244213 | 5.424471 | 2.50244213 | #N/A |
| 2.523275463 | 4.974746 | 2.523275463 | #N/A |
| 2.544108796 | 5.105621 | 2.544108796 | #N/A |
| 2.56494213 | 4.882367 | 2.56494213 | #N/A |
| 2.585775463 | 5.156937 | 2.585775463 | #N/A |
| 2.606608796 | 4.882068 | 2.606608796 | #N/A |
| 2.62744213 | 5.054303 | 2.62744213 | #N/A |
| 2.648275463 | 5.034556 | 2.648275463 | 6.109157 |
| 2.669108796 | 4.835382 | 2.669108796 | 5.83853 |
| 2.689953704 | 5.057273 | 2.689953704 | 6.071649 |
| 2.710787037 | 4.504433 | 2.710787037 | 6.257731 |
| 2.73162037 | 4.725886 | 2.73162037 | 5.978051 |
| 2.752453704 | 4.707865 | 2.752453704 | 5.687498 |
| 2.773275463 | 4.474821 | 2.773275463 | 5.510823 |
| 2.794108796 | 4.595435 | 2.794108796 | 5.745687 |
| 2.814953704 | 4.846455 | 2.814953704 | 5.493782 |
| 2.835787037 | 4.349487 | 2.835787037 | 5.420269 |
| 2.85662037 | 4.623514 | 2.85662037 | 5.677184 |
| 2.877453704 | 4.35981 | 2.877453704 | 5.499728 |
| 2.898287037 | 4.580013 | 2.898287037 | 5.273839 |
| 2.91912037 | 4.233418 | 2.91912037 | 5.523314 |
| 2.939953704 | 4.033472 | 2.939953704 | 5.601781 |
| 2.960787037 | 3.875247 | 2.960787037 | 5.556786 |
| 3.0009375 | 4.083802 | 3.0009375 | 5.661055 |
| 3.023287037 | 3.564172 | 3.023287037 | 5.20255 |
| 3.04412037 | 3.788096 | 3.04412037 | 5.251106 |
| 3.064953704 | 3.721753 | 3.064953704 | 5.24757 |
| 3.12525463 | 3.615655 | 3.12525463 | 5.073968 |
| 3.166898148 | 3.759606 | 3.166898148 | 5.125836 |
| 3.208564815 | 3.402011 | 3.208564815 | 5.700113 |
| 3.250231481 | 3.312303 | 3.250231481 | 5.346854 |
| 3.291898148 | 3.384652 | 3.291898148 | 5.366998 |
| 3.333553241 | 2.754262 | 3.333553241 | 5.469024 |
| 3.416898148 | 2.657981 | 3.416898148 | 4.906005 |
| 3.458564815 | 2.661131 | 3.458564815 | 4.953602 |
| 3.500231481 | 2.683549 | 3.500231481 | 5.018805 |
| 3.541909722 | 2.315241 | 3.541909722 | 5.040889 |
| 3.583564815 | 2.470533 | 3.583564815 | 4.669607 |
| 3.625243056 | 2.895316 | 3.625243056 | 4.677879 |
| 3.666909722 | 3.167133 | 3.666909722 | 4.748203 |
| 3.708564815 | 2.959319 | 3.708564815 | 4.306628 |
| 3.750243056 | 3.334286 | 3.750243056 | 4.003834 |
| 3.791898148 | 3.10766 | 3.791898148 | 4.363513 |
| 3.833587963 | 3.058263 | 3.833587963 | 4.014596 |
| 3.875243056 | 2.723771 | 3.875243056 | 4.028898 |
| 3.916909722 | 2.612081 | 3.916909722 | 4.080404 |
| 3.958576389 | 2.666911 | 3.958576389 | 3.442322 |
| 4.00025463 | 2.121485 | 4.00025463 | 3.755342 |
| 4.040208333 | 2.498356 | 4.040208333 | 3.691836 |
| 4.063460648 | 2.796938 | 4.063460648 | 3.801793 |
| 4.084293981 | 3.222628 | 4.084293981 | 3.397573 |
| 4.105127315 | 3.059871 | 4.105127315 | 3.198539 |
| 4.125960648 | 3.144483 | 4.125960648 | 6.444279 |
| 4.146793981 | 2.912629 | 4.146793981 | 6.634366 |
| 4.167627315 | 2.798553 | 4.167627315 | 6.147713 |
| 4.188460648 | 2.657885 | 4.188460648 | 6.247666 |
| 4.209305556 | 2.724152 | 4.209305556 | 6.187882 |
| 4.230127315 | 2.72257 | 4.230127315 | 6.114422 |
| 4.250960648 | 2.797554 | 4.250960648 | 5.93613 |
| 4.271793981 | 3.035758 | 4.271793981 | 5.516821 |
| 4.332094907 | 2.726879 | 4.332094907 | 5.486897 |
| 4.373726852 | 2.984358 | 4.373726852 | 5.457622 |
| 4.415405093 | 2.487146 | 4.415405093 | 5.381355 |
| 4.457060185 | 2.364557 | 4.457060185 | 5.195489 |
| 4.498738426 | 2.894607 | 4.498738426 | 4.731695 |
| 4.540393519 | 3.171245 | 4.540393519 | 4.725901 |
| 4.623738426 | 3.579278 | 4.623738426 | 4.398326 |
| 4.665405093 | 3.227408 | 4.665405093 | 4.601714 |
| 4.707071759 | 2.769516 | 4.707071759 | 3.739007 |
| 4.74875 | 3.303736 | 4.74875 | 4.125107 |
| 4.810706019 | 2.604359 | 4.810706019 | 3.918031 |
| 4.833958333 | 2.666446 | 4.833958333 | 3.87917 |
| 4.854791667 | 2.436089 | 4.854791667 | 3.812785 |
| 4.875625 | 2.365274 | 4.875625 | #N/A |
| 4.896458333 | 3.052339 | 4.896458333 | #N/A |
| 4.917291667 | 3.356655 | 4.917291667 | #N/A |
| 4.938125 | 3.536857 | 4.938125 | #N/A |
| 4.958958333 | 3.254377 | 4.958958333 | 8.184118 |
| 4.979803241 | 2.647855 | 4.979803241 | 7.679708 |
| 5.000625 | 2.479576 | 5.000625 | 7.4381 |
| 5.021458333 | 3.108576 | 5.021458333 | 6.956085 |
| 5.042291667 | 2.733165 | 5.042291667 | 6.785896 |
| 5.063136574 | 2.161332 | 5.063136574 | 6.765765 |
| 5.083958333 | 2.115124 | 5.083958333 | 6.793903 |
| 5.104803241 | 2.617033 | 5.104803241 | 6.765692 |
| 5.125636574 | 2.554023 | 5.125636574 | 6.222265 |
| 5.146458333 | 2.480167 | 5.146458333 | 6.749342 |
| 5.167291667 | 2.715101 | 5.167291667 | 5.725123 |
| 5.188136574 | 2.735876 | 5.188136574 | 5.549073 |
| 5.208969907 | 2.725627 | 5.208969907 | 5.06423 |
| 5.229803241 | 2.575811 | 5.229803241 | 5.338056 |
| 5.250636574 | 2.212894 | 5.250636574 | 5.471513 |
| 5.271458333 | 2.233998 | 5.271458333 | 5.151946 |
| 5.292303241 | 2.213399 | 5.292303241 | 5.546629 |
| 5.313136574 | 2.766555 | 5.313136574 | 5.259173 |
| 5.333969907 | 2.52938 | 5.333969907 | 4.601235 |
| 5.354803241 | 2.933614 | 5.354803241 | 4.772757 |
| 5.375636574 | 3.028033 | 5.375636574 | 4.52338 |
| 5.396469907 | 3.41555 | 5.396469907 | 4.513873 |
| 5.417303241 | 3.193063 | 5.417303241 | 4.173473 |
| 5.438136574 | 3.138092 | 5.438136574 | 3.831865 |
| 5.458981481 | 2.893515 | 5.458981481 | 3.9247 |
| 5.479814815 | 3.43812 | 5.479814815 | 3.336164 |
| 5.500636574 | 3.013834 | 5.500636574 | 3.628655 |
| 5.521469907 | 3.132246 | 5.521469907 | 3.92468 |
| 5.542314815 | 3.046817 | 5.542314815 | 7.176596 |
| 5.563148148 | 3.078321 | 5.563148148 | 6.633468 |
| 5.583981481 | 2.615919 | 5.583981481 | 6.08785 |
| 5.604803241 | 2.751108 | 5.604803241 | 6.244726 |
| 5.625636574 | 2.824868 | 5.625636574 | 5.927638 |
| 5.646469907 | 2.517154 | 5.646469907 | 7.42588 |
| 5.667314815 | 1.988747 | 5.667314815 | 6.687646 |
| 5.688148148 | 2.344756 | 5.688148148 | 7.307424 |
| 5.708969907 | 3.218347 | 5.708969907 | 6.437283 |
| 5.729814815 | 2.85646 | 5.729814815 | 5.960429 |
| 5.750648148 | 2.43488 | 5.750648148 | 6.032461 |
| 5.771493056 | 2.792278 | 5.771493056 | 6.137525 |
| 5.811608796 | 2.982295 | 5.811608796 | 6.469258 |
| 5.833981481 | 2.991141 | 5.833981481 | 6.484286 |
| 5.854814815 | 3.201134 | 5.854814815 | 5.838443 |
| 5.875659722 | 2.563264 | 5.875659722 | 5.693282 |
| 5.896481481 | 2.42295 | 5.896481481 | 6.134384 |
| 5.917314815 | 2.673206 | 5.917314815 | 5.663696 |
| 5.938148148 | 2.654685 | 5.938148148 | 5.459308 |
| 5.958981481 | 2.747516 | 5.958981481 | 5.10138 |
| 5.979814815 | 2.548837 | 5.979814815 | 5.754516 |
| 6.019282407 | 2.525679 | 6.019282407 | 4.844961 |
| 6.060914352 | 2.808173 | 6.060914352 | 5.415936 |
| 6.102592593 | 2.547346 | 6.102592593 | 5.179432 |
| 6.144259259 | 2.485466 | 6.144259259 | 4.849273 |
| 6.185925926 | 2.707999 | 6.185925926 | 4.904904 |
| 6.227592593 | 3.150225 | 6.227592593 | 4.450798 |
| 6.269259259 | 2.60164 | 6.269259259 | 4.495592 |
| 6.310925926 | 2.741736 | 6.310925926 | 3.395906 |
| 6.352592593 | 2.407971 | 6.352592593 | 4.206471 |
| 6.394259259 | 1.757518 | 6.394259259 | 3.473652 |
| 6.435925926 | 2.549188 | 6.435925926 | 3.669552 |
| 6.477604167 | 3.543268 | 6.477604167 | 8.226236 |
| 6.519270833 | 3.739929 | 6.519270833 | 8.798409 |
| 6.5609375 | 3.384398 | 6.5609375 | 8.077047 |
| 6.602604167 | 3.33986 | 6.602604167 | 7.873461 |
| 6.644270833 | 2.969001 | 6.644270833 | 7.76911 |
| 6.6859375 | 2.726888 | 6.6859375 | 7.415218 |
| 6.727604167 | 2.846601 | 6.727604167 | 6.526413 |
| 6.769270833 | 2.275316 | 6.769270833 | 6.82022 |
| 6.8109375 | 2.198233 | 6.8109375 | 6.822738 |
| 6.852615741 | 3.320418 | 6.852615741 | 6.629892 |
| 6.894282407 | 3.746778 | 6.894282407 | 6.207532 |
| 6.935949074 | 3.943445 | 6.935949074 | 6.731417 |
| 6.977615741 | 3.363937 | 6.977615741 | 5.485258 |
| 7.019282407 | 2.890475 | 7.019282407 | 6.309702 |
| 7.060949074 | 3.262214 | 7.060949074 | 5.860365 |
| 7.102615741 | 2.954454 | 7.102615741 | 5.880978 |
| 7.144282407 | 2.153391 | 7.144282407 | 5.84526 |
| 7.185960648 | 2.378666 | 7.185960648 | 5.735903 |
| 7.227662037 | 2.9512 | 7.227662037 | 5.541218 |
| 7.269293981 | 3.551366 | 7.269293981 | 5.192567 |
| 7.310960648 | 3.218829 | 7.310960648 | 9.177272 |
| 7.352627315 | 3.12968 | 7.352627315 | 8.703374 |
| 7.394293981 | 2.593928 | 7.394293981 | 8.983128 |
| 7.435960648 | 2.394028 | 7.435960648 | 8.965026 |
| 7.477627315 | 2.21824 | 7.477627315 | 8.120359 |
| 7.519293981 | 3.134434 | 7.519293981 | 8.137175 |
| 7.560960648 | 2.766007 | 7.560960648 | 8.314145 |
| 7.602627315 | 2.512249 | 7.602627315 | 8.698809 |
| 7.644305556 | 2.630357 | 7.644305556 | 8.641541 |
| 7.685972222 | 2.416168 | 7.685972222 | 8.071362 |
| 7.727638889 | 2.661644 | 7.727638889 | 8.489848 |
| 7.769305556 | 2.79807 | 7.769305556 | 8.062885 |
| 7.810960648 | 2.972875 | 7.810960648 | 7.448528 |
| 7.852638889 | 2.41065 | 7.852638889 | 8.106278 |
| 7.894305556 | 2.495323 | 7.894305556 | 7.770178 |
| 7.935972222 | 2.934737 | 7.935972222 | 8.291804 |
| 7.977638889 | 2.847816 | 7.977638889 | 7.42387 |
| 8.01931713 | 3.15902 | 8.01931713 | 8.205845 |
| 8.060983796 | 3.667069 | 8.060983796 | 7.910364 |
| 8.102638889 | 3.282952 | 8.102638889 | 7.724277 |
| 8.14431713 | 2.793275 | 8.14431713 | 7.616001 |
| 8.185983796 | 2.452958 | 8.185983796 | 7.379514 |
| 8.227650463 | 2.630365 | 8.227650463 | 7.477386 |
| 8.26931713 | 2.729709 | 8.26931713 | 6.807137 |
| 8.310983796 | 2.807003 | 8.310983796 | 6.842168 |
| 8.352650463 | 2.620657 | 8.352650463 | 9.308379 |
| 8.39431713 | 3.13093 | 8.39431713 | 8.968605 |
| 8.436030093 | 2.627208 | 8.436030093 | 9.14572 |
| 8.477662037 | 2.251114 | 8.477662037 | 8.747909 |
| 8.51931713 | 2.646687 | 8.51931713 | 8.726134 |
| 8.56099537 | 3.079137 | 8.56099537 | 8.391006 |
| 8.602662037 | 2.563705 | 8.602662037 | 8.450653 |
| 8.644328704 | 3.087527 | 8.644328704 | 7.990832 |
| 8.68599537 | 2.590317 | 8.68599537 | 8.18066 |
| 8.727662037 | 2.968817 | 8.727662037 | 7.942457 |
| 8.769340278 | 3.12238 | 8.769340278 | 7.713663 |
| 8.811006944 | 3.547524 | 8.811006944 | 8.415674 |
| 8.852673611 | 4.297379 | 8.852673611 | 7.626019 |
| 8.894340278 | 4.161104 | 8.894340278 | 8.069413 |
| 8.936018519 | 5.030762 | 8.936018519 | 8.045293 |
| 8.977673611 | 5.637126 | 8.977673611 | 8.527124 |
| 9.019351852 | 5.298599 | 9.019351852 | 7.610373 |
| 9.061006944 | 4.932112 | 9.061006944 | 7.099549 |
| 9.102685185 | 5.059932 | 9.102685185 | 7.573514 |
| 9.144351852 | 4.555223 | 9.144351852 | 7.538042 |
| 9.186018519 | 4.263374 | 9.186018519 | 7.441958 |
| 9.227685185 | 4.428963 | 9.227685185 | 7.639114 |
| 9.269351852 | 4.978399 | 9.269351852 | 6.761559 |
| 9.311018519 | 5.80515 | 9.311018519 | 7.284119 |
| 9.352685185 | 5.421699 | 9.352685185 | 7.794689 |
| 9.394351852 | 5.041867 | 9.394351852 | 9.245949 |
| 9.436018519 | 4.245652 | 9.436018519 | 10.85137 |
| 9.477685185 | 4.627719 | 9.477685185 | 10.59078 |
| 9.519363426 | 5.043918 | 9.519363426 | 10.01031 |
| 9.561030093 | 5.134606 | 9.561030093 | 9.805758 |
| 9.602696759 | 4.84806 | 9.602696759 | 10.12079 |
| 9.644398148 | 3.838338 | 9.644398148 | 10.16871 |
| 9.686030093 | 4.53542 | 9.686030093 | 9.679668 |
| 9.727696759 | 4.92595 | 9.727696759 | 9.62599 |
| 9.769351852 | 4.769973 | 9.769351852 | 9.378336 |
| 9.811030093 | 5.17225 | 9.811030093 | 10.05829 |
| 9.852696759 | 4.80986 | 9.852696759 | 8.640112 |
| 9.894363426 | 5.148977 | 9.894363426 | 9.457369 |
| 9.936030093 | 4.672589 | 9.936030093 | 9.403243 |
| 9.977708333 | 4.188494 | 9.977708333 | 9.422581 |
| 10.019375 | 4.707168 | 10.019375 | 9.496971 |
| 10.06104167 | 4.721385 | 10.06104167 | 8.947212 |
| 10.10269676 | 4.783384 | 10.10269676 | 8.878696 |
| 10.144375 | 4.512029 | 10.144375 | 9.005632 |
| 10.18604167 | 4.258463 | 10.18604167 | 8.788143 |
| 10.22770833 | 4.029292 | 10.22770833 | 8.814812 |
| 10.269375 | 4.322887 | 10.269375 | 8.966389 |
| 10.31104167 | 4.08165 | 10.31104167 | 8.892519 |
| 10.35265046 | 4.958148 | 10.35265046 | 9.361223 |
| 10.394375 | 5.847916 | 10.394375 | 8.628824 |
| 10.43604167 | 6.32333 | 10.43604167 | 8.199861 |
| 10.47770833 | 6.265306 | 10.47770833 | 7.797361 |
| 10.51938657 | 5.801625 | 10.51938657 | 8.34846 |
| 10.56104167 | 5.735916 | 10.56104167 | 9.992476 |
| 10.60271991 | 5.45328 | 10.60271991 | 10.55201 |
| 10.64438657 | 5.33565 | 10.64438657 | 10.78163 |
| 10.68605324 | 5.542859 | 10.68605324 | 10.40103 |
| 10.72771991 | 5.033404 | 10.72771991 | 9.900923 |
| 10.76938657 | 4.913043 | 10.76938657 | 9.858058 |
| 10.81105324 | 5.076824 | 10.81105324 | 10.93733 |
| 10.85275463 | 4.666098 | 10.85275463 | 10.56453 |
| 10.89439815 | 4.554989 | 10.89439815 | 10.63292 |
| 10.93605324 | 4.729548 | 10.93605324 | 10.1317 |
| 10.97771991 | 4.089445 | 10.97771991 | 10.15173 |
| 11.01938657 | 3.973743 | 11.01938657 | 10.03745 |
| 11.06105324 | 4.564354 | 11.06105324 | 9.908442 |
| 11.10273148 | 4.511001 | 11.10273148 | 9.87036 |
| 11.14439815 | 5.108614 | 11.14439815 | 10.1959 |
| 11.18606481 | 4.441917 | 11.18606481 | 9.519185 |
| 11.22773148 | 4.69673 | 11.22773148 | 9.621466 |
| 11.26939815 | 4.755281 | 11.26939815 | 10.03958 |
| 11.31106481 | 4.227083 | 11.31106481 | 8.765776 |
| 11.35273148 | 4.190309 | | |
| 11.39439815 | 4.416976 | | |
| 11.43606481 | 4.467027 | | |
| 11.47773148 | 5.739811 | | |
| 11.51939815 | 5.667678 | | |
| 11.56107639 | 5.399963 | | |
| 11.60273148 | 5.114323 | | |
| 11.64440972 | 5.493369 | | |
| 11.68607639 | 4.566129 | | |
| 11.72774306 | 4.238223 | | |
| 11.76940972 | 4.256388 | | |
| 11.81107639 | 3.624721 | | |
| 11.85274306 | 4.105767 | | |
| 11.89440972 | 5.08095 | | |
| 11.93607639 | 5.102737 | | |
| 11.97775463 | 5.012239 | | |

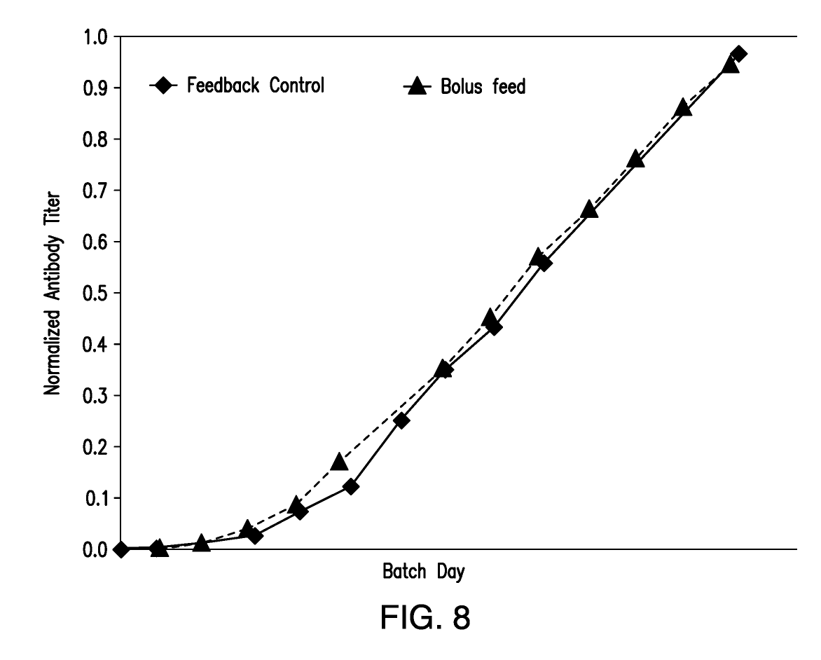

FIG. 8 shows the antibody titer for a feedback controlled continuous nutrient feed and for a bolus nutrient feed. As can be seen in FIG. 8, antibody production is unaffected by either method. Tables 4 and 5 below show the bolus fed antibody titer and feedback control antibody titer data points, respectively, for FIG. 8.

TABLE 4
BOLUS FED ANTIBODY TITER DATA POINTS FOR FIG. 8

| | Bolus feed | Bolus Fed |
|---|---|---|
| Bolus Feed Time | Ab Titer | Normalized |
| (Elapsed Days) | (mg/L) | Ab Titer |
| 0 | 0.866 | 0.000721667 |
| 0.831180556 | 2.362 | 0.001968333 |
| 1.668321759 | #N/A | #N/A |
| 2.614583333 | 32.606 | 0.027171667 |
| 3.625787037 | 89.425 | 0.074520833 |
| 4.531863426 | 148.02 | 0.12335 |
| 5.726122685 | 301.873 | 0.251560833 |
| 6.67775463 | 421.186 | 0.350988333 |
| 7.65849537 | 519.165 | 0.4326375 |
| 8.641284722 | 670.959 | 0.5591325 |
| 9.714537037 | #N/A | #N/A |
| 10.66090278 | #N/A | #N/A |
| 11.64418981 | #N/A | #N/A |
| 12.62819444 | 1158.82 | 0.965683333 |

TABLE 5
FEEDBACK CONTROL ANTIBODY TITER
DATA POINTS FOR FIG. 8

| Feedback Control | Feedback Control | Feedback Control |
|---|---|---|
| Time | Ab Titer | Normalized Ab Titer |
| 0 | #N/A | #N/A |
| 0.753171296 | 2.556 | 0.00213 |
| 1.749884259 | 15.36 | 0.0128 |
| 2.757048611 | 48.048 | 0.04004 |
| 3.710439815 | 105.017 | 0.087514167 |
| 4.757465278 | 205.669 | 0.171390833 |
| 5.814016204 | #N/A | #N/A |
| 6.735243056 | 423.018 | 0.352515 |
| 7.729918981 | 543.108 | 0.45259 |
| 8.767893519 | 683.645 | 0.569704167 |
| 9.742418981 | 795.66 | 0.66305 |
| 10.70917824 | 913.834 | 0.761528333 |
| 11.73123843 | 1034.809 | 0.862340833 |
| 12.79594907 | 1134.383 | 0.945319167 |

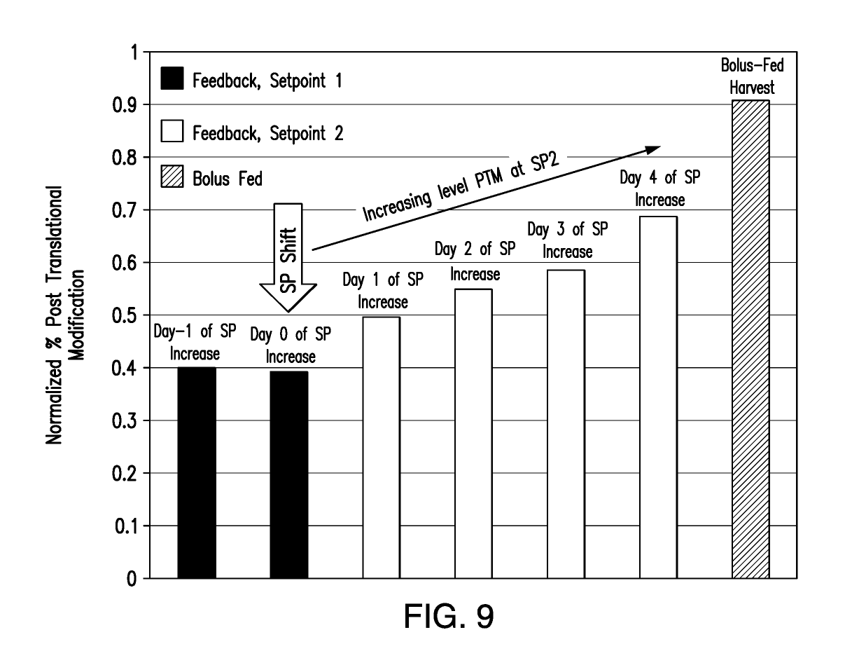

FIG. 9 shows the normalized percentage of PTM as a result of glucose concentration. As can be seen from FIG. 9, there is a decrease in PTM as the glucose concentration decreases from about 6 g/L-8 g/L (set point for bolus-fed harvest) to 5 g/L (set point 2) to 3 g/L (set point 1). In other words, lower exposure to nutrients results in a decrease in PTM. The data points in FIG. 9 for the normalized percentage of PTM are shown in Table 6 below.

TABLE 6
NORMALIZED % PTM DATA POINTS FOR FIG. 9

| | % Post Translational | Normalized % Post |
|---|---|---|
| Condition | Modification | Translational Modification |
| Day −1 of SP Increase | 12.03 | 0.401 |
| Day 0 of SP Increase | 11.79 | 0.393 |
| Day 1 of SP Increase | 14.88 | 0.496 |
| Day 2 of SP Increase | 16.48 | 0.549333333 |
| Day 3 of SP Increase | 17.58 | 0.586 |
| Day 4 of SP Increase | 20.63 | 0.687666667 |
| Bolus-Fed Harvest | 27.2 | 0.906666667 |

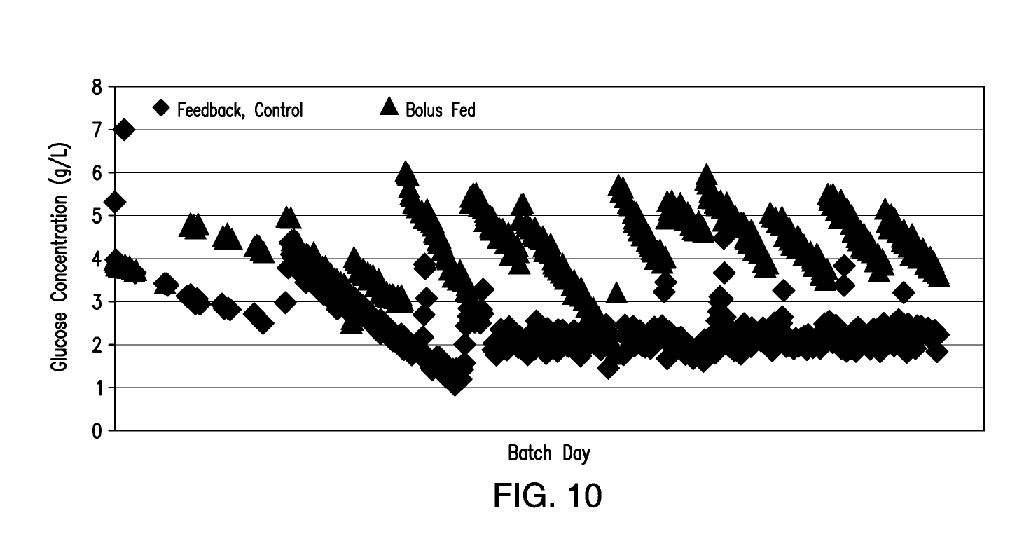

FIG. 10 shows the glucose concentrations for a feedback controlled continuous nutrient feed in accordance with the present invention and for a bolus nutrient feed. As can be seen by FIG. 10, the methods of the present invention are able to provide reduced, steady concentrations of glucose. The data points in FIG. 10 for the glucose concentrations are shown in Table 7 below.

TABLE 7
GLUCOSE CONCENTRATION DATA POINTS FOR FIG. 10

| | Glucose | | Glucose |
|---|---|---|---|
| Time (hrs) | Concentration | | Concentration |
| Feedback | Feedback Control | Time (hrs) | Bolus Fed |
| Control | (g/L) | Bolus Fed | (g/L) |
| 0 | 5.29985 | 0 | 3.9606 |
| 0.443888889 | 3.95717 | 0.443888889 | 3.92564 |
| 0.888055556 | 3.87786 | 0.888055556 | 3.82241 |
| 1.331944444 | 3.94245 | 1.554444444 | 3.84826 |
| 1.554444444 | 3.88536 | 1.998333333 | 3.78432 |
| 1.998333333 | 3.88327 | 2.442222222 | 3.81402 |
| 2.442222222 | 3.84436 | 4.382222222 | 3.83029 |
| 2.886111111 | 3.7485 | 5.334444444 | 3.75084 |
| 3.589444444 | 6.98909 | 6.226666667 | 3.80185 |
| 4.157777778 | 3.83584 | 7.119166667 | 3.72134 |
| 5.11 | 3.78798 | 8.011388889 | 3.68723 |
| 6.0025 | 3.7856 | 8.900833333 | 3.71741 |
| 6.894722222 | 3.73533 | 20.45444444 | 3.40678 |
| 7.787222222 | 3.68673 | 30.1175 | 4.74804 |
| 8.679444444 | 3.66978 | 31.01027778 | 4.80446 |
| 20.23388889 | 3.40307 | 31.90277778 | 4.76064 |
| 21.12222222 | 3.40884 | 32.79527778 | 4.69968 |
| 21.56944444 | 3.37754 | 33.68777778 | 4.7881 |
| 29.72194444 | 3.11293 | 43.51138889 | 4.50823 |
| 30.78583333 | 3.15921 | 44.40333333 | 4.44888 |
| 31.67833333 | 3.08833 | 45.295 | 4.56108 |
| 32.57111111 | 2.95089 | 46.18777778 | 4.44496 |
| 33.46333333 | 3.04687 | 47.07722222 | 4.43893 |
| 34.35305556 | 2.90941 | 56.46 | 4.27974 |
| 43.2875 | 2.92864 | 57.35194444 | 4.30659 |
| 44.17888889 | 2.81226 | 58.24444444 | 4.29294 |
| 45.07138889 | 2.85354 | 59.13638889 | 4.18843 |
| 45.96333333 | 2.83553 | 60.02611111 | 4.13743 |
| 46.85583333 | 2.79272 | 69.4075 | 4.95997 |
| 56.23555556 | 2.67934 | 71.02194444 | 4.9194 |
| 57.12805556 | 2.67136 | 71.46583333 | 4.41552 |
| 58.02 | 2.57063 | 71.90972222 | 4.38365 |
| 58.91194444 | 2.54624 | 72.35361111 | 4.42239 |
| 59.80472222 | 2.50303 | 73.02027778 | 4.31899 |
| 69.18361111 | 2.97555 | 73.46416667 | 4.37885 |
| 70.07583333 | 3.77294 | 73.90805556 | 4.3449 |
| 70.80111111 | 4.34847 | 74.35194444 | 4.23448 |
| 71.46583333 | 4.08935 | 75.01861111 | 4.24824 |
| 71.90972222 | 4.00212 | 75.4625 | 4.14202 |
| 72.35361111 | 3.99123 | 75.90638889 | 4.14761 |
| 72.7975 | 4.01331 | 76.35027778 | 4.07654 |
| 73.02027778 | 3.99191 | 77.01694444 | 4.04303 |
| 73.46416667 | 3.91424 | 77.46083333 | 4.10848 |
| 73.90805556 | 3.85688 | 77.90472222 | 4.02519 |
| 74.35194444 | 3.84475 | 78.34861111 | 3.97673 |
| 74.79583333 | 3.67941 | 79.01527778 | 3.97045 |
| 75.01861111 | 3.64752 | 79.45916667 | 3.99019 |
| 75.4625 | 3.66484 | 79.90305556 | 3.90772 |
| 75.90638889 | 3.6525 | 80.34694444 | 4.13212 |
| 76.35027778 | 3.55085 | 81.01361111 | 3.94071 |
| 76.79416667 | 3.45215 | 81.4575 | 3.93964 |
| 77.01694444 | 3.42771 | 81.90138889 | 3.93305 |
| 77.46083333 | 3.5292 | 82.34527778 | 3.90002 |
| 77.90472222 | 3.47243 | 83.01194444 | 3.78135 |
| 78.34861111 | 3.48275 | 83.45583333 | 3.80974 |
| 78.7925 | 3.44748 | 83.89972222 | 3.72092 |
| 79.01527778 | 3.51503 | 84.34361111 | 3.54584 |
| 79.45916667 | 3.40908 | 85.01055556 | 3.79766 |
| 79.90305556 | 3.4091 | 85.45472222 | 3.73607 |
| 80.34694444 | 3.40949 | 85.89861111 | 3.6327 |
| 80.79083333 | 3.37424 | 86.34277778 | 3.60241 |
| 81.01361111 | 3.66927 | 87.01 | 3.64506 |
| 81.4575 | 3.40708 | 87.45416667 | 3.4821 |
| 81.90138889 | 3.29053 | 87.89805556 | 3.49399 |
| 82.34527778 | 3.33054 | 88.34194444 | 3.50496 |
| 82.78916667 | 3.3244 | 89.00888889 | 3.53164 |
| 83.01194444 | 3.2331 | 89.45305556 | 3.31505 |
| 83.45583333 | 3.24332 | 89.89722222 | 3.27601 |
| 83.89972222 | 3.39759 | 90.34111111 | 3.33213 |
| 84.34361111 | 3.15861 | 91.00805556 | 3.43951 |
| 84.78777778 | 3.22317 | 91.45222222 | 3.38503 |
| 85.01055556 | 3.24632 | 91.89611111 | 3.1468 |
| 85.45472222 | 3.31019 | 92.34027778 | 3.4265 |
| 85.89861111 | 3.17534 | 93.00694444 | 3.24971 |
| 86.34277778 | 3.14291 | 93.45083333 | 3.19635 |
| 86.78694444 | 3.11793 | 93.895 | 3.27543 |
| 87.01 | 3.16349 | 94.33888889 | 3.09075 |
| 87.45388889 | 3.0751 | 95.24694444 | 2.49991 |
| 87.89805556 | 2.9869 | 95.69111111 | 2.57693 |
| 88.34194444 | 3.00619 | 96.135 | 2.5465 |
| 88.78583333 | 2.95103 | 96.57916667 | 4.02104 |
| 89.00888889 | 3.05399 | 97.02305556 | 3.98664 |
| 89.45305556 | 2.81784 | 97.46722222 | 3.95544 |
| 89.89694444 | 2.94564 | 97.91138889 | 3.86852 |
| 90.34111111 | 2.82913 | 98.35527778 | 3.66631 |
| 90.785 | 2.83378 | 99.0225 | 3.62051 |
| 91.00805556 | 2.91134 | 99.46638889 | 3.76868 |
| 91.45222222 | 3.09505 | 99.91027778 | 3.69577 |
| 91.89611111 | 2.86231 | 100.3544444 | 3.74638 |
| 92.34027778 | 2.95479 | 101.0216667 | 3.61072 |
| 92.78416667 | 2.84231 | 101.4655556 | 3.65232 |
| 93.00694444 | 2.81938 | 101.9094444 | 3.65673 |
| 93.45083333 | 2.79815 | 102.3536111 | 3.50981 |
| 93.895 | 2.83839 | 103.0205556 | 3.59905 |
| 94.33888889 | 2.93334 | 103.4647222 | 3.50056 |
| 95.02611111 | 2.94485 | 103.9086111 | 3.58028 |
| 95.69083333 | 3.01962 | 104.3525 | 3.51239 |
| 96.135 | 3.08518 | 105.0194444 | 3.35906 |
| 96.57888889 | 2.90996 | 105.4636111 | 3.46452 |
| 97.02305556 | 2.822 | 105.9077778 | 3.4217 |
| 97.46722222 | 2.60949 | 106.3516667 | 3.52777 |
| 97.91111111 | 2.98458 | 107.0186111 | 3.37968 |
| 98.35527778 | 2.99921 | 107.4627778 | 3.24786 |
| 98.79944444 | 2.89195 | 107.9066667 | 3.17432 |
| 99.02222222 | 2.88476 | 108.3508333 | 3.26832 |
| 99.46638889 | 2.80296 | 109.0180556 | 3.09402 |
| 99.91027778 | 2.81875 | 109.4619444 | 3.19621 |
| 100.3544444 | 2.88799 | 109.9061111 | 3.15208 |
| 100.7986111 | 2.7446 | 110.3502778 | 3.08408 |
| 101.0213889 | 2.71513 | 111.0169444 | 3.12704 |
| 101.4655556 | 2.62124 | 111.4611111 | 3.09169 |
| 101.9094444 | 2.7469 | 111.905 | 3.13017 |
| 102.3536111 | 2.6358 | 112.3488889 | 3.10825 |
| 102.7977778 | 2.64662 | 113.0161111 | 3.05118 |
| 103.0205556 | 2.64383 | 113.4602778 | 2.96148 |
| 103.4644444 | 2.48012 | 113.9041667 | 3.13752 |
| 103.9086111 | 2.56149 | 114.3483333 | 3.07076 |
| 104.3525 | 2.61773 | 115.0152778 | 2.97416 |
| 104.7966667 | 2.58291 | 115.4594444 | 3.11854 |
| 105.0194444 | 2.49816 | 115.9033333 | 3.01764 |
| 105.4636111 | 2.46984 | 117.2877778 | 6.00949 |
| 105.9075 | 2.5008 | 117.7316667 | 5.96736 |
| 106.3516667 | 2.47808 | 118.1758333 | 5.92612 |
| 106.7955556 | 2.24744 | 118.6194444 | 5.64293 |
| 107.0186111 | 2.57076 | 119.2863889 | 5.49402 |
| 107.4625 | 2.47027 | 119.7302778 | 5.43498 |
| 107.9066667 | 2.43396 | 120.1741667 | 5.47254 |
| 108.3508333 | 2.43259 | 120.6180556 | 5.28723 |
| 108.7947222 | 2.4977 | 121.2847222 | 5.26741 |
| 109.0177778 | 2.38829 | 121.7286111 | 5.17114 |
| 109.4619444 | 2.34725 | 122.1725 | 5.22748 |
| 109.9058333 | 2.22657 | 122.6163889 | 5.18455 |
| 110.35 | 2.27469 | 123.2830556 | 5.05853 |
| 110.7941667 | 2.3519 | 123.7269444 | 5.09368 |
| 111.0169444 | 2.28667 | 124.1708333 | 5.06618 |
| 111.4608333 | 2.29553 | 124.6147222 | 4.92785 |
| 111.905 | 2.30401 | 125.2813889 | 4.95126 |
| 112.3488889 | 2.1131 | 125.7252778 | 5.12272 |
| 112.7930556 | 2.05542 | 126.1694444 | 5.04657 |
| 113.0158333 | 2.15201 | 126.6133333 | 4.89878 |
| 113.46 | 2.15773 | 127.28 | 4.89227 |
| 113.9041667 | 2.1462 | 127.7236111 | 4.83168 |
| 114.3480556 | 2.0095 | 128.1675 | 4.73809 |
| 114.7922222 | 2.00685 | 128.6113889 | 4.62723 |
| 115.015 | 2.08611 | 129.2783333 | 4.56662 |
| 115.4591667 | 2.23016 | 129.7222222 | 4.5413 |
| 115.9033333 | 1.89489 | 130.1661111 | 4.39996 |
| 116.3475 | 2.03546 | 130.61 | 4.36069 |
| 117.0672222 | 2.11907 | 131.2766667 | 4.47573 |
| 117.7316667 | 2.10383 | 131.7205556 | 4.19303 |
| 118.1755556 | 1.91726 | 132.1644444 | 4.17655 |
| 118.6194444 | 1.93228 | 132.6083333 | 4.24852 |
| 119.0636111 | 1.78201 | 133.275 | 4.07631 |
| 119.2863889 | 1.90199 | 133.7188889 | 4.01898 |
| 119.7302778 | 1.76972 | 134.1627778 | 3.97811 |
| 120.1741667 | 1.81882 | 134.6066667 | 3.7236 |
| 120.6180556 | 1.90338 | 135.2736111 | 3.78111 |
| 121.0619444 | 1.86254 | 135.7177778 | 3.82847 |
| 121.2847222 | 1.89595 | 136.1613889 | 3.56015 |
| 121.7286111 | 1.95022 | 136.6052778 | 3.56488 |
| 122.1725 | 2.03028 | 137.2722222 | 3.59907 |
| 122.6163889 | 2.02368 | 137.7158333 | 3.53736 |
| 123.0602778 | 1.80358 | 138.1597222 | 3.51143 |
| 123.2830556 | 1.86305 | 138.6036111 | 3.48144 |
| 123.7269444 | 1.68852 | 139.2705556 | 3.69714 |
| 124.1708333 | 2.16485 | 139.7144444 | 3.53598 |
| 124.6147222 | 2.68219 | 140.1583333 | 3.56975 |
| 125.0586111 | 3.84445 | 140.6022222 | 3.46682 |
| 125.2813889 | 3.75849 | 141.5097222 | 3.27107 |
| 125.7252778 | 3.05046 | 141.9536111 | 3.37317 |
| 126.1691667 | 1.60889 | 142.3975 | 3.19992 |
| 126.6133333 | 1.55251 | 142.8413889 | 3.29018 |
| 127.0569444 | 1.49635 | 143.285 | 5.29681 |
| 127.28 | 1.4625 | 143.7288889 | 5.42912 |
| 127.7238889 | 1.5599 | 144.1730556 | 5.31815 |
| 128.1675 | 1.411 | 144.6169444 | 5.49514 |
| 128.6113889 | 1.59737 | 145.2836111 | 5.31922 |
| 129.0555556 | 1.49927 | 145.7275 | 5.50698 |
| 129.2783333 | 1.55528 | 146.1713889 | 5.40168 |
| 129.7222222 | 1.68831 | 146.6152778 | 5.21572 |
| 130.1661111 | 1.65586 | 147.2819444 | 5.22277 |
| 130.61 | 1.69803 | 147.7258333 | 5.32597 |
| 131.0538889 | 1.51503 | 148.1697222 | 5.25509 |
| 131.2766667 | 1.62337 | 148.6133333 | 5.18307 |
| 131.7205556 | 1.56305 | 149.0683333 | 5.08164 |
| 132.1644444 | 1.53581 | 149.3183333 | 4.88397 |
| 132.6083333 | 1.39492 | 149.5683333 | 5.06794 |
| 133.0522222 | 1.35263 | 149.8183333 | 5.01549 |
| 133.275 | 1.2922 | 150.0686111 | 4.91031 |
| 133.7188889 | 1.21502 | 150.3186111 | 4.92284 |
| 134.1627778 | 1.38027 | 150.5686111 | 4.88071 |
| 134.6066667 | 1.30947 | 150.8186111 | 4.90576 |
| 135.0505556 | 1.3538 | 151.0686111 | 4.7337 |
| 135.2736111 | 1.36581 | 151.3186111 | 4.98071 |
| 135.7175 | 1.19768 | 151.5686111 | 4.66753 |
| 136.1613889 | 1.41395 | 151.8186111 | 4.73602 |
| 136.6052778 | 1.08014 | 152.2625 | 4.67663 |
| 137.0494444 | 1.32496 | 152.7066667 | 4.66436 |
| 137.2719444 | 1.34268 | 153.1505556 | 4.79716 |
| 137.7158333 | 1.45098 | 153.5947222 | 4.70976 |
| 138.16 | 1.3088 | 154.0388889 | 4.68658 |
| 138.6038889 | 1.39873 | 154.4827778 | 4.45627 |
| 139.0475 | 1.36488 | 154.9269444 | 4.69575 |
| 139.2705556 | 1.19001 | 155.3711111 | 4.61841 |
| 139.7144444 | 1.40293 | 156.0380556 | 4.58039 |
| 140.1583333 | 1.41103 | 156.4822222 | 4.6775 |
| 140.6022222 | 1.5462 | 156.9263889 | 4.4771 |
| 141.2888889 | 2.01927 | 157.3702778 | 4.35384 |
| 141.9536111 | 2.42777 | 158.0372222 | 4.4401 |
| 142.3975 | 2.63074 | 158.4811111 | 4.56737 |
| 142.8413889 | 2.83209 | 158.9252778 | 4.42704 |
| 143.285 | 2.72224 | 159.3691667 | 4.07445 |
| 143.7288889 | 2.63608 | 160.0361111 | 4.36575 |
| 144.1730556 | 2.69195 | 160.4802778 | 4.13995 |
| 144.6169444 | 2.71345 | 160.9241667 | 4.22379 |
| 145.0608333 | 2.50984 | 161.3680556 | 4.17469 |
| 145.2836111 | 2.6369 | 162.035 | 4.28975 |
| 145.7275 | 2.60541 | 162.4788889 | 4.13539 |
| 146.1713889 | 2.67274 | 162.9230556 | 3.87281 |
| 146.6152778 | 2.69351 | 163.3672222 | 4.87836 |
| 147.0591667 | 2.50699 | 164.0338889 | 5.2242 |
| 147.2819444 | 2.68272 | 164.4780556 | 5.24807 |
| 147.7258333 | 2.80848 | 165.165 | 5.03418 |
| 148.1697222 | 2.71963 | 165.8297222 | 4.81739 |
| 148.6133333 | 3.27574 | 166.2736111 | 4.73886 |
| 152.2625 | 1.84522 | 166.7177778 | 4.87246 |
| 152.7066667 | 2.02054 | 167.1616667 | 4.77461 |
| 153.1505556 | 1.89572 | 167.6058333 | 4.68469 |
| 153.5947222 | 1.7493 | 168.2725 | 4.5802 |
| 154.0386111 | 1.82994 | 168.7166667 | 4.5102 |
| 154.4827778 | 2.03299 | 169.1608333 | 4.70917 |
| 154.9269444 | 1.84201 | 169.6047222 | 4.54906 |
| 155.3708333 | 2.33961 | 170.2716667 | 4.58545 |
| 155.815 | 2.17287 | 170.7155556 | 4.46504 |
| 156.0380556 | 2.09251 | 171.1597222 | 4.47254 |
| 156.4822222 | 2.00326 | 171.6036111 | 4.42642 |
| 156.9263889 | 2.00972 | 172.2705556 | 4.48492 |
| 157.3702778 | 1.95632 | 172.7147222 | 4.27087 |
| 157.8144444 | 1.85693 | 173.1586111 | 4.16092 |
| 158.0372222 | 1.87511 | 173.6027778 | 4.23464 |
| 158.4811111 | 2.25587 | 174.2697222 | 4.18793 |
| 158.9252778 | 2.41394 | 174.7138889 | 4.17626 |
| 159.3691667 | 2.27275 | 175.1580556 | 4.12183 |
| 159.8133333 | 2.33431 | 175.6022222 | 4.31591 |
| 160.0361111 | 2.11631 | 176.2691667 | 3.96654 |
| 160.48 | 2.15315 | 176.7130556 | 3.86951 |
| 160.9241667 | 2.21482 | 177.1572222 | 4.05681 |
| 161.3680556 | 2.10691 | 177.6013889 | 3.80757 |
| 161.8119444 | 1.9879 | 178.2683333 | 3.88444 |
| 162.0347222 | 2.07513 | 178.7122222 | 3.7184 |
| 162.4788889 | 2.09918 | 179.1563889 | 3.76801 |
| 162.9230556 | 2.045 | 179.6002778 | 3.65193 |
| 163.3669444 | 2.0579 | 180.2672222 | 3.8665 |
| 163.8111111 | 1.9786 | 180.7113889 | 3.60753 |
| 164.0338889 | 2.04415 | 181.1552778 | 3.56228 |
| 164.4780556 | 2.11519 | 181.5994444 | 3.51562 |
| 164.9219444 | 2.04256 | 182.2663889 | 3.53538 |
| 165.8297222 | 1.92716 | 182.7105556 | 3.58554 |
| 166.2736111 | 1.74054 | 183.1544444 | 3.52299 |
| 166.7177778 | 2.17775 | 183.5986111 | 3.50055 |
| 167.1616667 | 2.21902 | 184.2655556 | 3.35449 |
| 167.6055556 | 2.23581 | 184.7097222 | 3.15678 |
| 168.0497222 | 2.1295 | 185.1536111 | 3.49221 |
| 168.2725 | 2.06408 | 185.5977778 | 3.31856 |
| 168.7166667 | 1.95822 | 186.2644444 | 3.1794 |
| 169.1608333 | 1.87785 | 186.7086111 | 3.261 |
| 169.6047222 | 2.38464 | 187.1525 | 3.26585 |
| 170.0486111 | 2.52549 | 187.5963889 | 3.11678 |
| 170.2716667 | 2.48755 | 188.0405556 | 3.29677 |
| 170.7155556 | 2.39386 | 188.4847222 | 3.13789 |
| 171.1594444 | 2.26082 | 188.9288889 | 3.04174 |
| 171.6036111 | 2.10124 | 189.8586111 | 2.84437 |
| 172.0477778 | 2.04631 | 190.7886111 | 2.97215 |
| 172.2705556 | 1.96783 | 191.7183333 | 2.74657 |
| 172.7144444 | 2.03789 | 192.6480556 | 2.85061 |
| 173.1586111 | 1.96485 | 193.5780556 | 2.71859 |
| 173.6025 | 1.75977 | 194.5077778 | 2.64369 |
| 174.0466667 | 2.13635 | 195.4377778 | 2.23807 |
| 174.2697222 | 2.35361 | 196.3675 | 2.16861 |
| 174.7138889 | 2.19967 | 197.2975 | 2.18502 |
| 175.1577778 | 2.2276 | 198.2275 | 2.02487 |
| 175.6019444 | 2.26713 | 199.1572222 | 2.00279 |
| 176.0461111 | 2.27076 | 200.0861111 | 2.05927 |
| 176.2688889 | 2.08234 | 201.0158333 | 1.77877 |
| 176.7130556 | 2.05613 | 201.9455556 | 3.21063 |
| 177.1569444 | 1.98094 | 202.8752778 | 5.70505 |
| 177.6011111 | 2.09971 | 203.8047222 | 5.55309 |
| 178.0452778 | 2.13739 | 204.7341667 | 5.62934 |
| 178.2683333 | 1.81014 | 205.6636111 | 5.40796 |
| 178.7122222 | 2.33795 | 206.5933333 | 5.26706 |
| 179.1561111 | 2.27909 | 207.5230556 | 5.24844 |
| 179.6002778 | 2.13411 | 208.4522222 | 5.04861 |
| 180.0441667 | 2.28842 | 208.8961111 | 4.9106 |
| 180.2672222 | 2.3228 | 209.3402778 | 4.83827 |
| 180.7113889 | 2.20826 | 209.7844444 | 5.05838 |
| 181.1552778 | 2.1662 | 210.2286111 | 4.83412 |
| 181.5991667 | 1.97546 | 210.6725 | 4.76257 |
| 182.0433333 | 2.11621 | 211.1166667 | 4.64707 |
| 182.2661111 | 2.07917 | 211.7836111 | 4.80408 |
| 182.7102778 | 1.95 | 212.2275 | 4.53231 |
| 183.1544444 | 2.00555 | 212.6716667 | 4.68255 |
| 183.5983333 | 2.1972 | 213.1155556 | 4.661 |
| 184.0425 | 1.99805 | 213.7827778 | 4.53894 |
| 184.2652778 | 1.90735 | 214.2266667 | 4.38914 |
| 184.7094444 | 2.07147 | 214.6705556 | 4.51892 |
| 185.1536111 | 2.30457 | 215.1147222 | 4.35161 |
| 185.5975 | 1.94533 | 215.7816667 | 4.2933 |
| 186.0416667 | 2.04383 | 216.2255556 | 4.2022 |
| 186.2644444 | 2.02201 | 216.6697222 | 4.14232 |
| 186.7086111 | 2.00486 | 217.1136111 | 4.19824 |
| 187.1525 | 1.87491 | 217.7808333 | 3.98641 |
| 187.5963889 | 1.71041 | 218.225 | 4.17967 |
| 188.0405556 | 2.27353 | 218.6688889 | 4.12755 |
| 188.4844444 | 2.27361 | 219.1130556 | 3.98162 |
| 188.9286111 | 2.21939 | 219.7797222 | 4.18885 |
| 189.6158333 | 2.32112 | 220.2238889 | 3.99614 |
| 190.5455556 | 2.23684 | 220.6680556 | 3.88445 |
| 191.4752778 | 2.00438 | 221.1122222 | 4.00875 |
| 192.4052778 | 2.08773 | 221.7794444 | 4.02466 |
| 193.335 | 1.98721 | 222.2233333 | 4.92433 |
| 194.2647222 | 2.34499 | 222.6675 | 5.31792 |
| 195.1947222 | 2.07045 | 223.1116667 | 5.10258 |
| 196.1247222 | 1.87379 | 223.7786111 | 5.18651 |
| 197.9844444 | 2.44455 | 224.2227778 | 5.33129 |
| 198.9144444 | 1.43529 | 224.6669444 | 5.31647 |
| 199.8438889 | 2.10835 | 225.1111111 | 5.22186 |
| 200.7730556 | 2.16165 | 225.7780556 | 5.09756 |
| 201.7027778 | 2.03911 | 226.2219444 | 5.0919 |
| 202.6325 | 2.02224 | 226.6661111 | 5.09598 |
| 203.5619444 | 2.04709 | 227.1102778 | 5.20148 |
| 204.4916667 | 1.74866 | 227.7775 | 5.27139 |
| 205.4205556 | 2.42807 | 228.2213889 | 5.17647 |
| 206.3502778 | 2.3646 | 228.6655556 | 4.97104 |
| 207.28 | 2.29919 | 229.1097222 | 4.95102 |
| 208.2313889 | 2.37703 | 229.7766667 | 5.02617 |
| 208.8961111 | 2.39499 | 230.2208333 | 4.89217 |
| 209.3402778 | 1.97051 | 230.6647222 | 5.06075 |
| 209.7841667 | 2.24512 | 231.1088889 | 4.91127 |
| 210.2283333 | 2.25347 | 231.7758333 | 4.75924 |
| 210.6725 | 2.08371 | 232.6836111 | 4.86344 |
| 211.1166667 | 2.15365 | 233.1275 | 4.66869 |
| 211.5605556 | 2.29691 | 233.5713889 | 4.77352 |
| 211.7833333 | 2.03092 | 234.0155556 | 4.63601 |
| 212.2275 | 1.97129 | 234.4597222 | 4.71014 |
| 212.6716667 | 1.9721 | 234.9038889 | 4.69685 |
| 213.1155556 | 2.07924 | 235.3477778 | 4.83778 |
| 213.5597222 | 1.93054 | 235.7919444 | 4.73268 |
| 213.7825 | 2.09871 | 236.2358333 | 4.72232 |
| 214.2266667 | 2.01653 | 236.6797222 | 4.70191 |
| 214.6705556 | 1.97157 | 237.1238889 | 4.61924 |
| 215.1147222 | 2.08205 | 237.7908333 | 5.82279 |
| 215.5586111 | 2.20945 | 238.2347222 | 5.95289 |
| 215.7816667 | 1.90401 | 238.6788889 | 5.7376 |
| 216.2255556 | 2.25764 | 239.1227778 | 5.39835 |
| 216.6697222 | 2.20062 | 239.7897222 | 5.55047 |
| 217.1136111 | 2.38191 | 240.2336111 | 5.45566 |
| 217.5577778 | 2.30704 | 240.6777778 | 5.56575 |
| 217.7808333 | 2.3666 | 241.1219444 | 5.37954 |
| 218.225 | 2.21814 | 241.7888889 | 5.28663 |
| 218.6688889 | 2.22546 | 242.2330556 | 5.22091 |
| 219.1130556 | 2.29399 | 242.6769444 | 5.31419 |
| 219.5569444 | 2.35247 | 243.1211111 | 5.269 |
| 219.7797222 | 2.36244 | 243.7880556 | 5.33359 |
| 220.2238889 | 2.42202 | 244.2322222 | 5.20919 |
| 220.6677778 | 3.90842 | 244.6763889 | 5.16646 |
| 221.1119444 | 3.226 | 245.1202778 | 4.87647 |
| 221.5561111 | 3.24104 | 245.7872222 | 5.19865 |
| 221.7791667 | 3.44121 | 246.2313889 | 5.26332 |
| 222.2233333 | 2.10021 | 246.6755556 | 5.27455 |
| 222.6675 | 1.65588 | 247.1194444 | 4.9051 |
| 223.1116667 | 2.00054 | 247.7863889 | 4.96193 |
| 223.5555556 | 2.2584 | 248.2302778 | 4.95473 |
| 223.7786111 | 2.18337 | 248.6744444 | 4.87265 |
| 224.2227778 | 2.22002 | 249.1186111 | 4.88933 |
| 224.6666667 | 2.02996 | 249.7855556 | 4.95339 |
| 225.1108333 | 2.11005 | 250.2297222 | 4.91535 |
| 225.555 | 1.98403 | 250.6738889 | 4.8415 |
| 225.7780556 | 1.97535 | 251.1180556 | 4.73406 |
| 226.2219444 | 2.1047 | 251.785 | 4.75863 |
| 226.6661111 | 2.14528 | 252.2291667 | 4.83177 |
| 227.1102778 | 2.11167 | 252.6733333 | 4.73776 |
| 227.5541667 | 1.96546 | 253.1175 | 4.80804 |
| 227.7772222 | 2.1583 | 253.7841667 | 4.47607 |
| 228.2213889 | 2.09114 | 254.2283333 | 4.44379 |
| 228.6655556 | 2.03119 | 254.6722222 | 4.61578 |
| 229.1097222 | 2.03169 | 255.1163889 | 4.35294 |
| 229.5536111 | 1.805 | 255.7833333 | 4.35565 |
| 229.7766667 | 1.75306 | 256.4702778 | 4.63822 |
| 230.2208333 | 2.03753 | 257.4 | 4.1795 |
| 230.6647222 | 1.98862 | 258.3291667 | 4.3277 |
| 231.1088889 | 1.85836 | 259.2588889 | 4.10085 |
| 231.5530556 | 1.81241 | 260.1886111 | 4.15495 |
| 231.7758333 | 1.83977 | 261.1183333 | 3.90911 |
| 232.4627778 | 1.76471 | 262.0477778 | 3.81073 |
| 233.1275 | 1.63967 | 262.9772222 | 3.87842 |
| 233.5713889 | 1.79819 | 263.9061111 | 5.04643 |
| 234.0155556 | 1.74429 | 264.8347222 | 4.97527 |
| 234.4594444 | 1.77757 | 265.7641667 | 4.93942 |
| 234.9036111 | 1.82093 | 266.6936111 | 4.81825 |
| 235.3477778 | 1.75825 | 267.6225 | 4.80283 |
| 235.7919444 | 1.71644 | 268.2875 | 4.75164 |
| 236.2358333 | 1.64919 | 268.7313889 | 4.93642 |
| 236.6797222 | 1.65067 | 269.1755556 | 4.75401 |
| 237.1238889 | 1.59211 | 269.6197222 | 4.51092 |
| 237.5677778 | 2.09602 | 270.2866667 | 4.5984 |
| 237.7905556 | 2.0281 | 270.7308333 | 4.54899 |
| 238.2347222 | 2.0728 | 271.1747222 | 4.70999 |
| 238.6786111 | 1.96003 | 271.6188889 | 4.4307 |
| 239.1227778 | 2.13435 | 272.2858333 | 4.3134 |
| 239.5666667 | 2.14529 | 272.7297222 | 4.46242 |
| 239.7897222 | 2.12039 | 273.1738889 | 4.44403 |
| 240.2336111 | 2.1226 | 273.6177778 | 4.31874 |
| 240.6777778 | 2.17822 | 274.2847222 | 4.43482 |
| 241.1216667 | 2.09458 | 274.7286111 | 4.22428 |
| 241.5658333 | 1.93963 | 275.1727778 | 4.50794 |
| 241.7888889 | 1.78058 | 275.6166667 | 4.37905 |
| 242.2327778 | 1.92457 | 276.2836111 | 4.28183 |
| 242.6769444 | 2.54728 | 276.7277778 | 4.36293 |
| 243.1208333 | 2.7696 | 277.1716667 | 4.06209 |
| 243.565 | 2.96879 | 277.6155556 | 4.27271 |
| 243.7880556 | 3.0983 | 278.2825 | 4.05231 |
| 244.2322222 | 2.44977 | 278.7266667 | 4.19835 |
| 244.6761111 | 3.0513 | 279.1705556 | 4.10201 |
| 245.1202778 | 4.46037 | 279.6147222 | 4.0479 |
| 245.5641667 | 3.64992 | 280.2816667 | 4.14879 |
| 245.7872222 | 2.63717 | 280.7258333 | 4.01384 |
| 246.2311111 | 2.23246 | 281.1697222 | 3.94503 |
| 246.6752778 | 1.96177 | 281.6138889 | 3.82963 |
| 247.1194444 | 1.9733 | 282.2808333 | 4.01967 |
| 247.5633333 | 1.92291 | 282.725 | 4.08182 |
| 247.7863889 | 1.9421 | 283.1688889 | 3.83589 |
| 248.2302778 | 2.29655 | 283.6130556 | 3.8807 |
| 248.6744444 | 2.15675 | 284.28 | 3.60671 |
| 249.1186111 | 2.06017 | 284.7238889 | 3.74206 |
| 249.5625 | 1.83718 | 285.1680556 | 3.61191 |
| 249.7855556 | 2.26354 | 285.6119444 | 3.64284 |
| 250.2297222 | 2.15135 | 286.2788889 | 3.49373 |
| 250.6736111 | 2.13613 | 286.7227778 | 3.75384 |
| 251.1177778 | 2.01012 | 287.1666667 | 5.50193 |
| 251.5619444 | 1.91997 | 287.6108333 | 5.36619 |
| 251.785 | 2.04497 | 288.2777778 | 5.44722 |
| 252.2288889 | 1.76215 | 288.7219444 | 5.23718 |
| 252.6730556 | 1.91976 | 289.1661111 | 5.48611 |
| 253.1172222 | 2.17963 | 289.61 | 5.29237 |
| 253.5613889 | 2.49015 | 290.2769444 | 5.09807 |
| 253.7841667 | 2.31233 | 290.7211111 | 5.27902 |
| 254.2280556 | 2.25077 | 291.165 | 5.21127 |
| 254.6722222 | 2.41304 | 291.8522222 | 4.93468 |
| 255.1161111 | 2.32947 | 292.5169444 | 5.3445 |
| 255.5602778 | 2.27885 | 292.9611111 | 4.9385 |
| 255.7833333 | 1.94173 | 293.4052778 | 5.0282 |
| 256.2272222 | 2.39855 | 293.8491667 | 4.92491 |
| 257.1572222 | 1.97358 | 294.2933333 | 4.94234 |
| 258.0863889 | 2.13599 | 294.7375 | 5.14301 |
| 259.0161111 | 2.20439 | 295.1813889 | 5.09006 |
| 259.9458333 | 2.07312 | 295.6252778 | 4.97926 |
| 260.8752778 | 2.35689 | 296.2922222 | 4.79825 |
| 261.8052778 | 2.16814 | 296.7363889 | 4.98856 |
| 262.7347222 | 2.00509 | 297.1802778 | 4.64638 |
| 263.6638889 | 2.06753 | 297.6241667 | 4.83557 |
| 264.5925 | 1.85036 | 298.2913889 | 4.66544 |
| 265.5213889 | 2.46909 | 298.7352778 | 4.46933 |
| 266.4508333 | 2.44871 | 299.1794444 | 4.42644 |
| 267.3797222 | 2.44656 | 299.6233333 | 4.40905 |
| 268.2872222 | 2.48505 | 300.2902778 | 4.48562 |
| 268.7313889 | 2.63435 | 300.7344444 | 4.29635 |
| 269.1755556 | 3.24711 | 301.1786111 | 4.21742 |
| 269.6194444 | 2.23888 | 301.6227778 | 4.5645 |
| 270.0636111 | 2.07904 | 302.2894444 | 4.37116 |
| 270.2863889 | 2.21563 | 302.7333333 | 4.30076 |
| 270.7305556 | 1.91896 | 303.1775 | 4.357 |
| 271.1747222 | 2.09629 | 303.6216667 | 4.19275 |
| 271.6188889 | 2.04491 | 304.2888889 | 4.3476 |
| 272.0627778 | 1.96894 | 304.7327778 | 4.12702 |
| 272.2858333 | 2.10447 | 305.1766667 | 4.22847 |
| 272.7297222 | 1.98481 | 305.6208333 | 4.14018 |
| 273.1736111 | 1.88517 | 306.2877778 | 3.91622 |
| 273.6177778 | 2.02339 | 306.7319444 | 4.0101 |
| 274.0616667 | 2.1536 | 307.1758333 | 4.08905 |
| 274.2844444 | 1.94488 | 307.62 | 3.69736 |
| 274.7286111 | 2.09537 | 308.2866667 | 3.89877 |
| 275.1725 | 1.94546 | 308.7308333 | 3.91969 |
| 275.6166667 | 1.97124 | 309.1747222 | 3.95032 |
| 276.0605556 | 2.10351 | 309.6188889 | 3.83547 |
| 276.2833333 | 2.15169 | 310.2858333 | 5.15943 |
| 276.7275 | 2.06851 | 310.73 | 4.85061 |
| 277.1713889 | 1.95511 | 311.1738889 | 4.90129 |
| 277.6155556 | 2.17411 | 311.6177778 | 4.69627 |
| 278.0594444 | 1.91116 | 312.2847222 | 4.98669 |
| 278.2825 | 1.89503 | 312.7286111 | 4.99629 |
| 278.7263889 | 2.13133 | 313.1727778 | 4.92192 |
| 279.1705556 | 2.23375 | 313.6166667 | 4.91592 |
| 279.6147222 | 2.07922 | 314.2838889 | 4.79179 |
| 280.0588889 | 2.15941 | 314.7277778 | 4.82191 |
| 280.2816667 | 2.10306 | 315.1719444 | 4.62895 |
| 280.7258333 | 2.09977 | 315.6158333 | 4.68028 |
| 281.1697222 | 1.90922 | 316.5236111 | 4.39498 |
| 281.6138889 | 1.97935 | 316.9675 | 4.51145 |
| 282.0577778 | 1.98323 | 317.4116667 | 4.64399 |
| 282.2808333 | 2.13178 | 317.8558333 | 4.35246 |
| 282.725 | 2.05535 | 318.5230556 | 4.39085 |
| 283.1688889 | 2.17687 | 318.9669444 | 4.51255 |
| 283.6130556 | 2.10914 | 319.4111111 | 4.26767 |
| 284.0569444 | 1.88863 | 319.855 | 4.41338 |
| 284.28 | 1.90439 | 320.5225 | 4.04934 |
| 284.7238889 | 2.27687 | 320.9663889 | 4.54584 |
| 285.1680556 | 2.27819 | 321.4105556 | 4.21321 |
| 285.6119444 | 1.97363 | 321.8547222 | 4.26114 |
| 286.0561111 | 2.474 | 322.5219444 | 4.16462 |
| 286.2788889 | 2.08995 | 322.9658333 | 4.03369 |
| 286.7227778 | 2.25392 | 323.41 | 4.07753 |
| 287.1666667 | 2.16887 | 323.8541667 | 4.06638 |
| 287.6108333 | 2.53164 | 324.5213889 | 4.03094 |
| 288.0547222 | 2.19634 | 324.9652778 | 4.01644 |
| 288.2777778 | 2.18478 | 325.4094444 | 4.21972 |
| 288.7219444 | 2.05544 | 325.8536111 | 4.08692 |
| 289.1661111 | 2.28481 | 326.5205556 | 3.84686 |
| 289.61 | 2.18665 | 326.9644444 | 4.04213 |
| 290.0538889 | 2.44092 | 327.4086111 | 3.77223 |
| 290.2769444 | 2.30768 | 327.8527778 | 3.9225 |
| 290.7208333 | 2.09997 | 328.52 | 3.99757 |
| 291.165 | 2.13653 | 328.9638889 | 3.76221 |
| 291.6091667 | 2.29461 | 329.4080556 | 3.68814 |
| 292.5166667 | 1.89174 | 329.8522222 | 3.89506 |
| 293.405 | 3.35168 | 330.5194444 | 3.79475 |
| 293.8491667 | 3.81949 | 330.9636111 | 3.69956 |
| 294.2933333 | 1.83112 | 331.4075 | 3.64703 |
| 294.7372222 | 1.8642 | 331.8516667 | 3.57235 |
| 295.1813889 | 2.16381 | | |
| 295.6252778 | 2.17022 | | |
| 296.0691667 | 1.98928 | | |
| 296.2919444 | 1.90433 | | |
| 296.7361111 | 2.24558 | | |
| 297.1802778 | 2.07294 | | |
| 297.6241667 | 2.00742 | | |
| 298.0680556 | 2.04407 | | |
| 298.2911111 | 1.82856 | | |
| 298.7352778 | 2.18444 | | |
| 299.1791667 | 2.38328 | | |
| 299.6233333 | 1.94764 | | |
| 300.0675 | 2.35273 | | |
| 300.2902778 | 2.10771 | | |
| 300.7344444 | 2.18582 | | |
| 301.1783333 | 2.28062 | | |
| 301.6225 | 2.18726 | | |
| 302.0666667 | 2.01366 | | |
| 302.2894444 | 2.08052 | | |
| 302.7333333 | 2.115 | | |
| 303.1775 | 2.1862 | | |
| 303.6213889 | 2.23513 | | |
| 304.0655556 | 1.88516 | | |
| 304.2886111 | 2.01393 | | |
| 304.7327778 | 2.13416 | | |
| 305.1766667 | 1.95372 | | |
| 305.6205556 | 2.34303 | | |
| 306.0647222 | 2.20315 | | |
| 306.2877778 | 2.26925 | | |
| 306.7316667 | 2.10713 | | |
| 307.1758333 | 2.12814 | | |
| 307.62 | 2.36701 | | |
| 308.0636111 | 2.16943 | | |
| 308.2866667 | 1.82948 | | |
| 308.7308333 | 2.26683 | | |
| 309.1747222 | 2.1141 | | |
| 309.6188889 | 2.49 | | |
| 310.0630556 | 2.27842 | | |
| 310.2858333 | 2.20096 | | |
| 310.73 | 2.17509 | | |
| 311.1738889 | 2.15439 | | |
| 311.6177778 | 2.33172 | | |
| 312.0616667 | 2.19789 | | |
| 312.2847222 | 2.15463 | | |
| 312.7286111 | 2.27852 | | |
| 313.1725 | 1.99785 | | |
| 313.6166667 | 1.96589 | | |
| 314.0608333 | 2.49224 | | |
| 314.2836111 | 2.40053 | | |
| 314.7277778 | 2.37773 | | |
| 315.1716667 | 2.46324 | | |
| 315.6158333 | 2.55963 | | |
| 316.3027778 | 2.42319 | | |
| 317.4113889 | 3.20763 | | |
| 317.8558333 | 4.52655 | | |
| 318.2997222 | 2.16813 | | |
| 318.5227778 | 1.901 | | |
| 318.9669444 | 1.79215 | | |
| 319.4111111 | 2.46673 | | |
| 319.855 | 2.19232 | | |
| 320.2991667 | 2.22674 | | |
| 320.5222222 | 2.16041 | | |
| 320.9663889 | 2.30146 | | |
| 321.4102778 | 2.35759 | | |
| 321.8544444 | 2.06147 | | |
| 322.2986111 | 2.2465 | | |
| 322.5216667 | 1.90065 | | |
| 322.9658333 | 2.42279 | | |
| 323.41 | 2.29138 | | |
| 323.8541667 | 2.21841 | | |
| 324.2980556 | 2.42145 | | |
| 324.5211111 | 2.35336 | | |
| 324.9652778 | 2.25286 | | |
| 325.4094444 | 2.25769 | | |
| 325.8536111 | 2.31652 | | |
| 326.2975 | 2.24343 | | |
| 326.5205556 | 2.28121 | | |
| 326.9644444 | 2.32713 | | |
| 327.4086111 | 2.38217 | | |
| 327.8527778 | 2.14074 | | |
| 328.2966667 | 2.30334 | | |
| 328.5197222 | 2.2444 | | |
| 328.9638889 | 2.10546 | | |
| 329.4080556 | 2.16617 | | |
| 329.8522222 | 2.30982 | | |
| 330.2961111 | 2.12672 | | |
| 330.5191667 | 2.19646 | | |
| 330.9633333 | 1.81375 | | |
| 331.4075 | 2.20783 | | |

### Example 2: Comparison of Feedback Control and Bolus Fed Strategy

Materials and Methods

Cells were cultured under feedback control or bolus fed strategy as described above.

Results

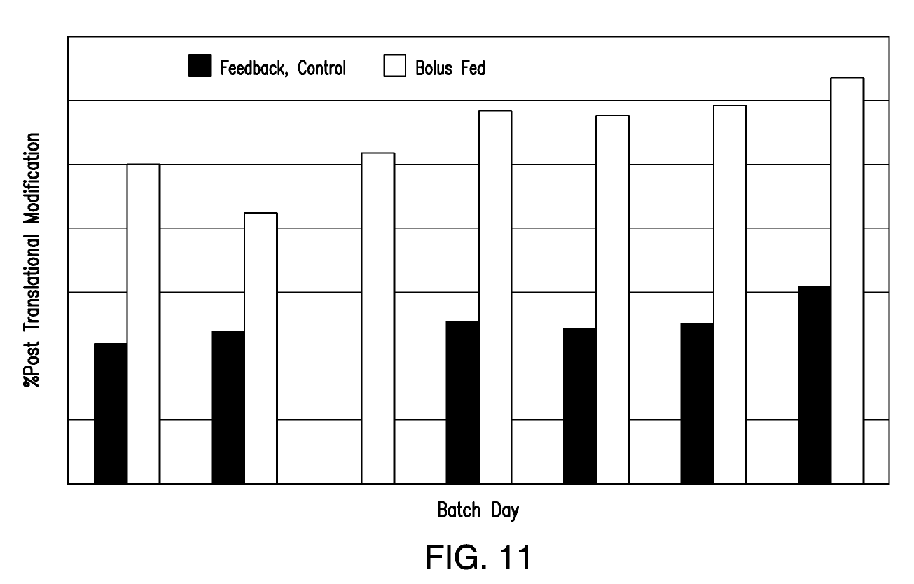

FIG. 11 shows the difference in PTMs in cells cultured using the feedback control or bolus fed strategy. Each pair of columns represents a batch day. For each pair of columns, the left column is the feedback control data, and the right column is the bolus fed data. The feedback control strategy (left column for each pair of columns) was confirmed to reduce the level of % PTM in the subsequent experiment as compared to the bolus feed strategy (right column for each pair of columns). Controlling the nutrient set point at a constant level, the % PTM was steadily maintained over the course of the production. The % PTM was also reduced from the bolus feed strategy thus demonstrating the ability to control antibody quality through Raman Spectroscopy feedback control.

The disclosed feedback control culture systems and methods provide real-time multi-component analysis without sample removal. Real time data enables automatic feedback control for continuous nutrient addition. Reduced, steady bioreactor concentrations of reactive nutrients results in lower level of antibody PTM by over 50% from standard bolus nutrient feed thus improving product quality and consistency.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

What is claimed is:

1. A method for controlling cell culture medium conditions comprising:
quantifying one or more analytes in the cell culture medium using in situ Raman spectroscopy; and
adjusting the one or more analyte concentrations in the cell culture medium to match predetermined analyte concentrations that maintain post-translational modifications of proteins in the cell culture medium to 1.0 to 30 percent.

2. The method of claim 1, wherein the post-translational modification comprises glycation.

3. The method of claim 1, wherein proteins in the cell culture comprise an antibody or antigen-binding fragment thereof.

4. The method of claim 1, wherein proteins in the cell culture comprise a fusion protein.

5. The method of claim 1, wherein the cell culture medium comprises mammalian cells.

6. The method of claim 5, wherein the mammalian cells comprise Chinese Hamster Ovary cells.

7. The method of claim 1, wherein the analyte is glucose.

8. The method of claim 7, wherein the predetermined glucose concentration is 0.5 to 8.0 g/L.

9. The method of claim 7, wherein the glucose concentration is 1.0 g/L to 3.0 g/L.

10. The method of claim 7, wherein the glucose concentration is 2.0 g/L.

11. The method of claim 7, wherein the glucose concentration is 1.0 g/L.

12. The method of claim 1, wherein the predetermined analyte concentrations maintain post-translation modifications of proteins in the cell culture medium to 1.0 to 20 percent.

13. The method of claim 1, wherein the predetermined analyte concentrations maintain post-translation modifications of proteins in the cell culture medium to 5.0 to 10 percent.

14. The method of claim 1, wherein the quantifying of analytes is performed continuously.

15. The method of claim 1, wherein the quantifying of analytes is performed intermittently.

16. The method of claim 1, wherein the quantifying of analytes is performed in intervals.

17. The method of claim 1, wherein the quantifying of analytes is performed in 5 minute intervals.

18. The method of claim 1, wherein the quantifying of analytes is performed in 10 minute intervals.

19. The method of claim 1, wherein the quantifying of analytes is performed in 15 minute intervals.

20. The method of claim 1, wherein the quantifying of analytes is performed hourly.

21. The method of claim 1, wherein the quantifying of analytes is performed at least daily.

22. The method of claim 1, wherein the adjusting of analyte concentrations is performed automatically.

23. The method of claim 1, wherein at least two different analytes are quantified.

24. The method of claim 1, wherein at least three different analytes are quantified.

25. The method of claim 1, wherein at least four different analytes are quantified.

26. A method for reducing post-translation modifications of a secreted protein comprising:
culturing cells secreting the protein in a cell culture medium comprising 0.5 to 8.0 g/L glucose;
incrementally determining the concentration of glucose in the cell culture medium during culturing of the cells using in situ Raman spectroscopy;
adjusting the glucose concentration to maintain the concentration of glucose to 0.5 to 8.0 g/L by automatically delivering multiple doses of glucose per hour to maintain post-translational modifications of the secreted protein to 1.0 to 30.0 percent.

27. The method of claim 26, wherein the concentration of glucose is 1.0 to 3.0 g/L.

28. A system for controlling cell culture medium conditions comprising:
one or more processors in communication with a computer readable medium storing software code for execution by the one or more processors in order to cause the system to
receive data comprising a concentration of one or more analytes in the cell culture medium from an in situ Raman spectrometer; and
adjust the one or more analyte concentrations in the cell culture medium to match predetermined analyte concentrations that maintain post-translational modifications of proteins in the cell culture medium to 1.0 to 30 percent.

29. The system of claim 28, wherein the software code is further configured to cause the system to perform chemometric analysis on the data.

30. The system of claim 29, wherein the chemometric analysis comprises Partial Least Squares regression modeling.

31. The system of claim 28, wherein the software code is further configured to cause the system to perform one or more signal processing techniques on the data.

32. The system of claim 31, wherein the signal processing technique comprises a noise reduction technique.

33. A system for reducing post-translation modifications of a secreted protein comprising:
one or more processors in communication with a computer readable medium storing software code for execution by the one or more processors in order to cause the system to
incrementally receive spectral data comprising a concentration of glucose in a cell culture medium during culturing of cells secreting the protein from an in situ Raman analyzer; and
adjust the glucose concentration to maintain the concentration of glucose to 0.5 to 8.0 g/L by automatically delivering multiple doses of glucose per hour to maintain post-translational modifications of the secreted protein to 1.0 to 30.0 percent.

34. The system of claim 33, wherein the software code is further configured to cause the system to correlate peaks within the spectral data to glucose concentrations.

35. The system of claim 33, wherein the software code is further configured to perform Partial Least Squares regression modeling on the spectral data.

36. The system of claim 33, wherein the software code is further configured to perform a noise reduction technique on the spectral data.

37. The system of claim 33, wherein the adjustment of the glucose concentration is performed by automated feedback control software.

38. The system of claim 33, wherein the concentration of glucose is 1.0 to 3.0 g/L.
